(19) United States
(12) Patent Application Publication
(10) Pub. No.: US 2021/0145921 A1
(43) Pub. Date: May 20, 2021

(54) COMPOSITION COMPRISING VEGF ANTAGONISTS AND A CATIONIC PEPTIDE AND USES THEREOF

(71) Applicant: UCL BUSINESS LTD, London (GB)

(72) Inventors: Francesca CORDEIRO, London (GB); Benjamin Michael DAVIS, London (GB); Ljuban GRGIC, London (GB); Satyanarayana SOMAVARAPU, London (GB)

(21) Appl. No.: 16/641,076

(22) PCT Filed: Aug. 23, 2018
(86) PCT No.: PCT/GB2018/052400

§ 371 Date: Feb. 21, 2020

(30) Foreign Application Priority Data
Aug. 25, 2017 (GB) 1713724.1

(51) Int. Cl. A61K 38/08; A61K 39/395; C07K 16/22; A61K 47/26; A61K 9/06; A61K 47/36; A61K 9/00

(52) U.S. Cl. CPC A61K 38/08; A61K 39/3955; C07K 16/22; A61K 47/26; C07K 2317/24; A61K 47/36; A61K 9/0048; C07K 2317/76; A61K 9/06

(57) ABSTRACT

The invention relates compositions comprising a small cationic peptide and a VEGF antagonist, and the use of such compositions in the treatment or prevention of conditions characterised by VEGF dysfunction, for example vasculoproliferative conditions, particularly in the eye.

### FIELD OF THE INVENTION

The invention relates to the use of a composition comprising VEGF antagonists and the R8 peptide for use in use in the treatment or prevention of vasculoproliferative conditions, particularly in the eye.

### BACKGROUND OF THE INVENTION

Anti-Vascular Endothelial Growth Factors (anti-VEGFs) have revolutionised the management of age-related macular degeneration (AMD). Ranibizumab (Lucentis) intraocular injections are the most common National Institute for Health and Care Excellence (NICE)-approved anti-VEGF treatments, although successful injection regimens are very expensive (National Health Service (NHS) estimate £85 million/year). In addition to the increasing health-service burden, injections are associated with significant ocular complications including endophthalmitis, retinal detachment, cataract and uveitis, and are unappealing to patients. There is a wealth of clinical data supporting the efficacy and safety of Lucentis. Like the other monoclonal antibody treatments in this category it presently requires administration by a specialist clinician via intravitreal injection which is unpleasant for the patient and can be associated with infection, bleeding and retinal detachment.

### SUMMARY OF THE INVENTION

The present invention describes a novel formulation to dramatically enhance the amount of anti-VEGF reaching intraocular tissues via topical administration. It makes use of a small cationic peptide (e.g. R8, oligoarginine, RRRRRRRR (SEQ ID NO: 1)). Until now, R8 has been characterized as a cell-penetrating peptide (CPP). CPPs are defined as peptides which allow passage across cell membrane barriers allowing intracellular uptake of drugs (Ye et al. Int. J. Mol. Sci. 2016, 17(11), 1892). Our studies confirm that R8 promotes corneal uptake in vivo and in vitro, where passage of Lucentis was demonstrated through an intact cellular membrane barrier.

However, a surprising finding was that R8 promoted anti-VEGF delivery through corneal cells. This was demonstrated using a transcytosis assay and measuring the change in drug permeability coefficient (Pₐₚₚ) of Avastin and Lucentis in the presence of R8/ST-R8. Pₐₚₚ of both anti-VEGFs was considerably increased in the presence of R8/ST-R8. This enhancement was found to occur in a concentration-dependent manner, with a molar ratio of between 40:1 and 200:1 R8 to antagonist being effective, and a molar ratio of between 50:1 to 80:1 R8 to antagonist being particularly effective.

Further investigation showed that passage across the corneal cells was non-toxic with R8 at all concentrations studied, and only toxic with ST(Stearyl)-R8 when the molar ratio >40:1. No changes were recorded in the transepithelial resistance (TEER), indicating involvement of a transcellular mechanism, as the corneal barrier integrity remained intact. This is again a new finding as previous studies have suggested that poly-arginine (36 kDa) polypeptides induced reversible disruption of tight junctions manifested as a change in TEER (Nemoto et al. Biol Pharm Bull. 2007 September; 30(9):1768-72.). Additionally, these assays clearly showed that presence of R8 does not interfere with the VEGF binding activity. Finally, further functional assessment of anti-VEGF activity was performed in vivo in rabbits where only one eye was dosed with topical instillation of 10 mg/mL Lucentis compared to 10 mg/mL Lucentis/50 mg/mL R8. Measurements at 2 hours after topical application showed R8 to significantly increase levels of Lucentis in the vitreous and retina/choroid of freshly enucleated eyes (10 v 215 ng/g tissue).

The unique aspects of this approach is its simplicity as it does not require complex formulation or expensive constituents; simply mixing lyophilised peptide with VEGF antagonist such as Lucentis at the optimal molar ratio enables delivery of therapeutically relevant concentration to posterior segment tissues.

The invention uses Lucentis (Ranibizumab) in a preferred embodiment, and includes a novel formulation enabling administration as an eye-drop. This will:

Avoid the unpleasant patient experience and potential adverse effects of intravitreal injection

Enable self-administration at home

Avoid costs associated with clinic visits for treatment and management of injection-related adverse effects

Enable a potential dosing advantage (and potentially lower cost of therapy).

Half-life of ranibizumab in the eye is reported to be 7.19 days. A high dose must therefore be used in order to maintain a therapeutic concentration at the back of the eye for the full month prior to the next injection.

The addition of small cationic peptides (like R8) to other drug proteins (antibodies, antibody fragments, therapeutic proteins) would similarly enhance the ability of these factors to be transported across biological barriers in a similar manner to that presented for Lucentis.

Our studies indicate that ˜70% of the absorbed topically applied dose reaches intraocular tissues from topical rather than systemic absorption—a highly desirable pharmacodynamic property. No reports of ocular irritation or injury were recorded from rabbit in vivo studies and both agents were well tolerated using an in vitro model of the corneal epithelium (HCE-S cells).

Aggregation of the R8 and VEGF antagonist in the composition of the invention was reduced by the addition of Trehalose to the formulation. A concentration of 600 mg/ml Trehalose was found to be particularly effective.

Finally, in addition to high corneal permeation, enhancing ocular drug contact time is a recognised strategy for enhancing delivery of topically applied drugs to posterior ocular tissues. In the present invention enhancement of ocular drug contact time was achieved by formulating the aforementioned anti-VEGF and peptide mixture with a viscosity enhancing agent such as a polymer or hydrogel. Incorporation of the anti-VEGF/R8 combination in a thermosetting hydrogel comprising 20% (w/v) Lutrol F127, dramatically enhanced the delivery of anti-VEGF to posterior ocular tissues compared to the previously described liquid eye drop formulation. The inventors propose that this effect will not be limited to Lutrol-F127 containing hydrogels and would also be observed for other bio-compatible viscosity enhancers, combinations thereof (e.g. hyaluronic acid, hypermellose, etc) or other biocompatible hydrogel gel-forming materials.

The inventors have also found that the addition of a mucoadhesive polymer, such as chitosan, to the formulation enhances the delivery of the formulation to posterior ocular tissues.

Thus, the invention provides:

A composition comprising a small cationic peptide and a VEGF antagonist, wherein the molar ratio of small cationic peptide to VEGF antagonist is at least 40:1.

The invention also provides:

A method of prevention or treatment of a condition characterised by VEGF dysfunction, said method comprising administering a prophylactically or therapeutically effective amount of the composition as defined herein to a patient in need thereof.

The invention also provides:

A method of prevention or treatment of a vasculoproliferative condition of the eye, said method comprising administering a prophylactically or therapeutically effective amount of the composition as defined herein to a patient in need thereof.

The invention also provides:

The composition of the invention as defined herein, for use in the prevention or treatment of a condition characterised by VEGF dysfunction.

The invention also provides:

A kit comprising the composition of the invention as defined herein.

### BRIEF DESCRIPTION OF THE DRAWINGS

FIG. 1—In-vitro measurement of transcellular passage of anti-VEGF

Transwell assay allows assessment of anti-VEGF transcytosis using TER as a measure of barrier integrity. TEER—Transepithelial electrical resistance describes the electrical resistance of the HCE-S corneal epithelial barrier. If the barrier remains intact, this value will not decline over the course of an experiment.

[A] Illustration of assay used. [B] Results demonstrating that the presence of R8 (Stearyl conjugated, ST-R8) enhanced the delivery of Lucentis across this corneal barrier model. (Two-way ANOVA with Bonferroni post-hoc tests **p<0.01, ***p<0.001).

FIG. 2—Addition of R8 and Lucentis has no effect on corneal epithelial barrier integrity (no TEER decline)

FIG. 3—A ratio of R8 to Lucentis of 40:1 or more significantly enhances corneal transcytosis in vitro

A surprising finding in our in vitro studies was that there is evidence that R8 promoted delivery across corneal cells. Another method to monitor the extent of delivery across corneal barrier models is to calculate the drug permeability coefficient (Pₐₚₚ) as described in Davis et al 2014. R8 (=>40:1 ratio) enhances Lucentis transcytosis (*p<0.05, **p<0.01). The effect of the ratio is surprising as previous work (de Cogan et al, 2017) has suggested a 25:1 molar ratio of R7 to Avastin is sufficient.

FIG. 4—R8 enhances delivery of VEGF antagonists across corneal barriers in vitro in a concentration dependent manner.

The presence of R8 (ST-R8) aided the transport of the anti-VEGFs [A] Avastin and [B] Lucentis across the HCE-S barrier (two tailed T-test, P=0.0096). [C] Stearyl-R8 enhanced the delivery of the anti-VEGF Lucentis across the HCE-S barrier in a concentration dependent manner. One-way ANOVA with Bonferroni's Multiple Comparison Test P=0.0307.

FIG. 5—HCE-S Toxicity assay with R8/Lucentis

R8 and Lucentis (maximal 200/1 ratio) combinations were well tolerated by HCE-S cells in vitro at all concentrations tested. Table shows molar ratios of R8 to Lucentis used at each point of the assay.

FIG. 6—R8 does not adversely influence VEGF₁₆₅ binding

A functional ELISA was used to monitor the affinity of Lucentis in the presence of varying concentrations of R8.

Addition of R8 to Lucentis did not negatively impact VEGF binding activity.

FIG. 7—Effect of R8 on VEGF₁₆₅ binding Avastin

A functional ELISA was used to monitor the affinity of Avastin in the presence of varying concentrations of R8.

Addition of R8 to Avastin did not negatively impact VEGF binding activity.

FIG. 8—Effect of R8:Lucentis ratio on stability at 25° C., and with the addition of Trehalose

Absorbance (600 nm) was used to assess R8:Lucentis (R/L) formulation aggregation over time.

The most stable formulations had a R/L ratio of 50 to 100/1.

Surprisingly, aggregation was delayed in the >100:1 R/L, formulations by increasing trehalose concentration (100 to 300 mg/mL tested).

FIG. 9—Accelerated stability study (6 hours 25° C. then 7 days 4° C.)

Increasing Trehalose (to 600 mg/mL) or reducing Lucentis concentration (1 mg/mL while maintaining R/L, ratio) increased stability.

FIG. 10—R8/Lucentis is stable for 3 months at 4° C.

[A] R8/Lucentis combinations at ratios of 40:1 and 80:1 are stable for 3 months at 4° C.

[B] Stability of 12 weeks formulation at 4° C. in 1:50 Lucentis:R8 (solution, not hydrogel). A Lucentis and R8 formulation 1:50 ratio combination remained stable after 12 weeks when stored at 4° C.

FIG. 11—ELISA detection of Lucentis for in vivo work

R8, though a highly charged peptide, does not interfere with the Lucentis detection ELISA used for the transwell assay.

FIG. 12—Rabbit experiment: Free Lucentis (2 h)

Low levels (˜20 ng) of free Lucentis were detected in retina 2 h (previously established Cₘₐₓ) after topical instillation. This data was used as controls to compare with the formulation data. The concentration detected in plasma was minimal (<0.1 ng/mL). The proportion of topically and systemically delivered drug can be estimated using the following equation;

$$Topical absorbed ( \% ) = 100 * \frac{D}{( D + C )}$$

where D is the concentration of Lucentis detected in the dosed eye tissue and C is the concentration in the co-eye. The proportion of dosed free Lucentis reaching the retina by the topical route was ˜30% (n=10).

FIG. 13—R8:Lucentis at a ratio of 20/1 does not enhance delivery to retina after 2 h

100 mg/mL Lucentis with 50 mg/mL R8, no significant improvement in Lucentis delivery to the retina compared to Lucentis only control (n=3) (16+/−6 ng/g versus 18+/−8 ng/g). This ratio of R peptide to anti-VEGF to is similar to that reported in the de Cogan (2017) reference (25:1).

FIG. 14—R8: Lucentis at a ratio of 200/1 enhances delivery to retina after 2 h

10 mg/mL Lucentis with 50 mg/mL R8, significant improvement in Lucentis delivery to the retina compared to Lucentis only control.

FIG. 15—Summary of Rabbit experiments

In vivo assessment of Lucentis concentrations in the retina after topical application of R/L, formulations at 2 h. No significant difference in retinal Lucentis level when free Lucentis control is compared with 20/1 R/L, ratio group. However, there is a significant increase (×13.5 fold) in retinal level when 200/1 R/L, ratio group is compared with 20/1 R/L group.

FIG. 16—Preparation of R8:Lucentis hydrogels to enhance delivery

To increase corneal contact time, R8:Lucentis formulations were made in with hydrogels to enhance formulation viscosity.

Lutrol F127 was chosen as it is already used in several existing clinical applications due to its thermosensitive properties.

The range of Lutrol F127 concentrations was between 15%-25% (w/v). Solutions were prepared by mixing the desired quantities of Lutrol F127. Between 20% and 25% Lutrol F127 was found to generate a thermosensitive gel with the desired properties.

FIG. 17—Accelerated stability study (25° C. then 4° C.)

[A] 20% Lutrol hydrogel improves stability of Lucentis formulations. The presence of Lutrol helps prevent aggregation of R8:Lucentis formulations (as seen by increased OD) even at 200:1 ratio.

[B] 12 week old formulation of U8:Lucentis Hydrogel showed good stability when stored at 4° C. (1:50 Lucentis:R8 ratio).

[C] Accelerated stability testing of U8:Lucentis (50:1 ratio) 12h at 25° C. showed no significant loss of VEGF binding activity (functional ELISA) compared to U8:Lucentis stored at 4° C. or a Lucentis only control.

[D] Stability of chitosan-containing U8:Lucentis formulations at the 4 week time-point. Stability of the U8:Lucentis (80:1) formulation with 0.1% w/v chitosan showed good stability for 4 weeks as determined by EC₅₀ ratio (U8:Lucentis/Standard) of less than 2.

FIG. 18—Sustained release was proven in vitro using a Lucentis hydrogel release assay

[A] (20% Lutrol gel) takes ˜1 h to liberate Lucentis. Similar results were obtained in vivo (Rabbit) with FITC loaded Lutrol hydrogels.

[B] Stability summary of R8/Lucentis liquid and hydrogel formulations

FIG. 19—Formulation of R8/Lucentis hydrogel 200:1 ratio (20% Lutrol) dramatically enhances delivery to retina

[A] Lucentis gel 2 h (200:1 ratio), n=3, Lucentis 10 mg/ml and R8 50 mg/ml. Significant concentrations of Lucentis were delivered to the retina vs undosed eye. Concentration reached 41823+/−18709 ng/g retinal tissue, exceeding target of 200 ng/g.

[B] Lucentis gel 12 h (200:1 ratio), n=3, Lucentis 10 mg/ml and R8 50 mg/ml. Significant concentrations of Lucentis were delivered to the retina vs undosed eye. Concentration reached 5282+/−2840 ng/g retinal tissue, exceeding target of 200 ng/g.

[C] 200:1 R8: Lucentis hydrogel formulation summary of retinal delivery over time.

FIG. 20—Formulation of R8/Lucentis hydrogel 50:1 ratio (20% Lutrol) dramatically enhances delivery to retina

[A] Lucentis gel 2 h (50:1 ratio), n=6, Lucentis 10 mg/ml and R8 12.5 mg/ml. Significant concentrations of Lucentis were delivered to the retina vs undosed eye. Concentration reached 1254+/−460 ng/g retinal tissue, exceeding target of 200 ng/g.

[B] Lucentis gel 2 h (50:1 ratio), n=6, Lucentis 10 mg/ml and R8 12.5 mg/ml. Significant concentrations of Lucentis were delivered to the retina vs undosed eye. Concentration reached 13790+/−6725 ng/g retinal tissue, exceeding target of 200 ng/g.

[C] Lucentis gel 2 h (50:1 ratio), n=6, Lucentis 10 mg/ml and R8 12.5 mg/ml. Significant concentrations of Lucentis were delivered to the retina vs undosed eye. Concentration reached 169+/−140 ng/g retinal tissue, below target of 200 ng/g.

[D] 50:1 R8: Lucentis hydrogel formulation summary of retinal delivery over time.

FIG. 21—Summary of hydrogel work and tolerability and toxicity of hydrogel formulation

[A] In vivo assessment of Lucentis concentrations in the retina after topical application of R/L, formulations at 2 h, 12 h, 24 h and 72 h. No significant difference in retinal Lucentis level when free Lucentis control was compared with 20/1 R/L ratio group at 2 h. However, there was a significant increase (×13.5 fold) in retinal level when 200/1 R/L, ratio group was compared with 20/1 R/L, group at 2 h. Furthermore, there was a ×195 fold increase retinal Lucentis level at 2 h, retained at 12 h, when the 200/1 Hydrogel formulation was compared with the 200/1 ratio group. There was also a significant increase in retinal level when 50/1 R/L ratio group was compared with 20/1 R/L group. In addition, further improvements in retinal delivery and retention were seen by increasing the R/L, ratio to 80:1 and adding the mucoadhesive chitosan. An increase in the R/L ratio to 80:1 and the addition of a mucoadhesive (0.1% chitosan) were found to substantially improve delivery versus previous iterations.

[B] Rabbit eyes corneal toxicity assessment. Looking for evidence of redness, swelling, discharge, ulceration, haemorrhaging or scarring in the treated eye. There was no evidence of corneal toxicity or evidence of redness or corneal damage associated with U8:Lucentis administration (as seen by fluorescein staining).

FIG. 22—Optimisation of formulation

Formulations were tested in accelerated stability assays at 25° C., 3 month stability assays at 4° C., in the HCE-S transwell in vitro assay and in rabbit studies in vivo. A ratio range of R8 to Lucentis of 50-80:1 was found to be optimal.

FIG. 23—Size Exclusion Chromatography to investigate binding of R8 to Lucentis

This chromatographic method is used for separation of molecules by size, or in some cases molecular weight. It is usually applied to large molecules or macromolecular complexes. There was no co-elution of Lucentis and FITC-R8, so no binding.

FIG. 24—Quartz Crystal Microbalance to investigate binding of R8 to Lucentis

This method measures a mass variation per unit area by measuring the change in frequency of a quartz crystal resonator. The resonance is disturbed by the addition or removal of a small mass due to oxide growth/decay or film deposition at the surface of the acoustic resonator. 10 μg/ml Lucentis used, approx. 100 μl injections. No binding to R8 was detected.

FIG. 25—Surface Plasmon Resonance to investigate binding of R8 to Lucentis

Lucentis immobilised on CM5 chip surface RU=3700. Some binding of R8 to Lucentis was detected.

FIG. 26—Isothermal titration calorimetry (ITC) to investigate binding of R8 to Lucentis

10 mM HisCl, 10% Trehalose, pH 5.5

[A] R8:Lucentis molar ratio

[B] R8 heats of dilution (ratios plotted just for illustration purposes)

[C] ITC results with 0-2.5 ratio of R8:Lucentis and 2.5-180 ratio of R8:Lucentis. No binding was detected.

FIG. 27—The transport of dextrans used to investigate whether R8 can carry dextrans across the membrane

R8 made no difference to the transport of different sizes of dextran across a membrane.

FIG. 28—Summary of investigations of interaction between R8 and Lucentis

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “small cationic peptide” includes “small cationic peptides”, and the like. “R8: Lucentis”, “R8/Lucentis” or “R8 to Lucentis” are used interchangeably to mean the ratio of R8 to Lucentis.

The terms “U8 Lucentis”, “U8 gel Lucentis” and “U8 Lucentis Hydrogel” are used interchangeably to mean a formulation of R8 and Lucentis that contains a hydrogel and trehalose.

All publications, patents and patent applications cited herein, whether supra or infra, are hereby incorporated by reference in their entirety.

The composition of the invention comprises a VEGF antagonist and a small cationic peptide. In preferred embodiments of the invention the small cationic peptide is R8. In preferred embodiments of the invention the VEGF antagonist is Lucentis. Thus, in a preferred embodiment the composition comprises a combination of R8 and Lucentis.

### VEGF Antagonist

VEGF antagonists of the invention can be defined as any agent that prevents or inhibits the expression or function of VEGF. VEGF antagonists of the invention can be proteins. VEGF antagonists of the invention can be a soluble receptor of VEGF, such as Aflibercept (Eylea), or an anti-VEGF antibody. VEGF antagonists of the invention can be diabodies or bispecific antibodies. VEGF antagonists of the invention can be fragments or derivatives of anti-VEGF antibodies. Examples of antibody fragments or derivatives include a Fab fragment, a F(ab′)₂ fragment, a Fab′ fragment, a Fd fragment, a Fv fragment, a dAb fragment and an isolated complementarity determining region (CDR). Single chain antibodies such as scFv antibodies are also intended to be encompassed. Thus VEGF antagonists of the invention can be scFv antibodies. VEGF antagonists of the invention can be anti-VEGF antibodies such as Bevacizumab (Avastin) or Ranibizumab (Lucentis). A preferred VEGF antagonist of the invention is Lucentis.

### Small Cationic Peptide

Small cationic peptides from part of the composition of the invention. Small cationic peptides of the invention may be cell penetrating peptides (CPP). Small cationic peptides of the invention aid the uptake of substances into tissues. Small cationic peptides of the invention aid the intracellular update of substances. Small cationic peptides of the invention enhance the transcytosis of substances. Intracellular update and transcytosis can be measured by the skilled person by methods known in the art. For example, transcytosis can be measured by an in vitro HCE-S transcytosis assay (as described in Davis et al (2014)).

Small cationic peptides may consist of 6, 7, 8, 9, 10, 11, 12, 13, 14, 15, 16, 17, 18, 19 or 20 amino acids and have an overall positive charge due to the presence of cationic amino acids. Cationic amino acids have a positive charge at physiological pH. Examples of such positively charged amino acids include Lysine, Histidine and Arginine.

It will be understood that small cationic peptides of the invention are intended to encompass certain variants of said peptides. Other than the engineering to reduce dimer formation discussed above, other modifications to the native amino acid sequence of a peptide may be made. For example to one, two, three, four, or five amino acids in the sequence of a peptide. Any such modifications will typically be conservative, in that if an amino acid in the native sequence is replaced with a different amino acid, the different amino acid will typically have similar properties to the native amino acid. The table below shows the properties of amino acids. Molecular weights are shown alongside the 3-letter code for each amino acid. The molecular weights given are those of the neutral, free amino acids; residue weights can be obtained by subtraction of one equivalent of water (18 g/mol). The invention also includes peptides containing N-and C-terminals modified or blocked to reduce or inhibit degradation by exopeptidase enzymes.

| | | | |
|---|---|---|---|
| Ala 89 | Aliphatic, hydrophobic, neutral | Met 149 | hydrophobic, neutral |
| Cys 121 | polar, hydrophobic, neutral | Asn 132 | polar, hydrophilic, neutral |
| Asp 133 | polar, hydrophilic, charged (−) | Pro 115 | hydrophobic, neutral |
| Glu 147 | polar, hydrophilic, charged (−) | Gln 146 | polar, hydrophilic, neutral |
| Phe 165 | Aromatic, hydrophobic, neutral | Arg 174 | polar, hydrophilic, charged (+) |
| Gly 75 | Aliphatic, neutral | Ser 105 | polar, hydrophilic, neutral |
| His 155 | aromatic, polar, hydrophilic, | Thr 119 | polar, hydrophilic, neutral |
| | charged (+) | | |
| Ile 131 | Aliphatic, hydrophobic, neutral | Val 117 | aliphatic, hydrophobic, neutral |
| Lys 146 | polar, hydrophilic, charged(+) | Trp 204 | aromatic, hydrophobic, neutral |
| Leu 131 | Aliphatic, hydrophobic, neutral | Tyr 181 | aromatic, polar, hydrophobic |

The residue or residues which are modified may be comprised in any part of the sequence.

Small cationic peptides of the invention aid the uptake of VEGF antagonists into tissues. These peptides aid the uptake of VEGF antagonists into the retina when the composition of the invention is topically administered to the eye. These peptides aid the transcytosis of VEGF antagonists to reach the retina when the composition of the invention is topically administered to the eye. In a preferred embodiment of the invention the small cationic peptide comprises an R8 peptide, or a modified R8 peptide (e.g. Stearyl-R8 (ST-R8)) or a peptide based on the R8 peptide. R8 is defined as being eight consecutive arginine resides, RRRRRRRR (SEQ ID NO:1).

In the composition of the invention, the ratio of small cationic peptide to VEGF antagonist is set out as a molar ratio. In the composition of the invention, the ratio of small cationic peptide to VEGF antagonist is at least 40:1, at least 50:1, at least 60:1, at least 70:1, at least 80:1, at least 90:1, at least 100:1, at least 150:1 or at least 200:1. A preferred ratio range of small cationic peptide to VEGF antagonist is 50:1 to 80:1. In the composition of the invention, the ratio of R8 to Lucentis is at least 40:1, at least 50:1, at least 60:1, at least 70:1, at least 80:1, at least 90:1, at least 100:1, at least 150:1 or at least 200:1. A preferred ratio range of R8 to Lucentis is 50:1 to 80:1.

### Peptide Synthesis

The small cationic peptides of the invention may be synthesised using methods well known in the art. Preferred methods include solid-phase peptide synthesis techniques and most preferably an automated or semiautomated peptide synthesizer. Typically, using such techniques, an α-N-carbamoyl protected amino acid and an amino acid attached to the growing peptide chain on a resin are coupled at room temperature in an inert solvent such as dimethylformamide, N-methylpyrrolidinone or methylene chloride in the presence of coupling agents such as dicyclohexylcarbodiimide and 1-hydroxybenzotriazole in the presence of a base such as diisopropyl-ethylamine. The α-N-carbamoyl protecting group is removed from the resulting peptide-resin using a reagent such as trifluoroacetic acid or piperidine, and the coupling reaction repeated with the next desired N-protected amino acid to be added to the peptide chain. Suitable N-protecting groups are well known in the art, and include t-butyloxycarbonyl (tBoc) and fluorenylmethoxycarbonyl (Fmoc).

The term “peptide” includes not only molecules in which amino acid residues are joined by peptide (—CO—NH—) linkages but also molecules in which the peptide bond is reversed. Such retro-inverse peptidomimetics may be made using methods known in the art, for example such as those described in Meziere et al (1997) J. Immunol. 159, 3230-3237. This approach involves making pseudopeptides containing changes involving the backbone, and not the orientation of side chains. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N-or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH₂ replaced with —NH(Me) or —N(Me)₂) (methylation). Another potential modification is that hydrogens of a hydroxyl group of an amino acid such as K can be replaced with an acetyl group (CH₃CO) (acetylation).

Analogues of peptides according to the invention may also include peptide variants that increase or decrease the peptide's half-life in vivo. Examples of analogues capable of increasing the half-life of peptides used according to the invention include peptoid analogues of the peptides, D-amino acid derivatives of the peptides, and peptide-peptoid hybrids. A further embodiment of the variant polypeptides used according to the invention comprises D-amino acid forms of the polypeptide. The preparation of polypeptides using D-amino acids rather than L-amino acids greatly decreases any unwanted breakdown of such an agent by normal metabolic processes, decreasing the amounts of agent which needs to be administered, along with the frequency of its administration.

### Formulations

In a preferred embodiment, the composition of the invention may also comprise other components. Such components may act to improve the stability of the formulation, by for example preventing aggregation, or may act to improve its efficacy. Such components may act to improve the stability of the formulation so that it is generally more stable during storage and transport and has a longer shelf-life than the equivalent formulation without the additional component.

In a preferred embodiment of the invention the composition of the invention also comprises Trehalose. Trehalose acts to delay aggregation of the small cationic peptide and VEGF antagonist formulation. The concentration of Trehalose in the composition can be 100 mg/ml or more, 200 mg/ml or more, 300 mg/ml or more, 400 mg/ml or more, 500 mg/ml or more or preferably 600 mg/ml or more. In a preferred embodiment of the invention the composition of the invention also comprises Trehalose at a concentration of approximately 100 mg/ml.

Other alternative or additional agents which can act to improve the stability of compositions include trehalose sugar, buffers, tonicity agents, simple carbohydrates, sugars such as sorbitol or sucrose, carbohydrate polymers, amino acids, oligopeptides, polyamino acids, polyhydric alcohols and ethers thereof, detergents, lipids, surfactants, antioxidants, salts, human serum albumin, gelatins, formaldehyde, polysorbate 80 or combinations thereof.

In a preferred embodiment of the invention, the composition of the invention comprises an agent that increases the viscosity of the composition. Viscosity can be measure by the skilled person by methods known in the art, for example by using a viscometer or rheometer. An example of an agent that increases viscosity is a hydrogel. In a preferred embodiment of the invention, the composition of the invention comprises a hydrogel. The hydrogel improves the contact time of the composition with the tissue of interest, for example the cornea, by increasing the viscosity of the composition. Common ingredients in hydrogels include polyvinyl alcohol, sodium polyacrylate, acrylate polymers and copolymers with an abundance of hydrophilic groups. Natural hydrogel materials are being investigated for tissue engineering; these materials include agarose, methylcellulose, hyaluronan, and other naturally derived polymers.

Alternative agents that can be added to the composition to increase the viscosity of the composition include Lutrol (Poloxamer) Chitosan, gellants, polysaccharides, proteins, polyethylene glycol and carbomers.

The hydrogel concentration in the composition of the invention may be between 15% and 25% w/v. Preferably, the concentration of hydrogel is between 20% and 25% w/v. The hydrogel can be, for example, hyaluronic acid, hypermellose, or another biocompatible hydrogel gel-forming material. In a preferred embodiment the hydrogel is Lutrol F127.

In a preferred embodiment, the composition of the invention comprises both Trehalose and a hydrogel. In a preferred embodiment, the composition of the invention comprises R8 and Lucentis at a ratio of at least 40:1, Trehalose and a hydrogel. In a preferred embodiment, the composition of the invention comprises R8 and Lucentis at a ratio of at least 50:1, Trehalose and a hydrogel. In a preferred embodiment, the composition of the invention comprises R8 and Lucentis at a ratio of 50:1 to 80:1, Trehalose and a hydrogel. In a preferred embodiment, the composition of the invention comprises R8 and Lucentis at a ratio of at least 100:1, Trehalose and a hydrogel. In a preferred embodiment, the composition of the invention comprises R8 and Lucentis at a ratio of at least 200:1, Trehalose and a hydrogel.

In a preferred embodiment, the composition of the invention comprises R8 and Lucentis at a ratio of at least 40:1, Trehalose at a concentration of at least 100 mg/ml and a hydrogel. In a preferred embodiment, the composition of the invention comprises R8 and Lucentis at a ratio of at least 50:1, Trehalose at a concentration of at least 100 mg/ml and a hydrogel. In a preferred embodiment, the composition of the invention comprises R8 and Lucentis at a ratio of 50:1 to 80:1, Trehalose at a concentration of at least 100 mg/ml and a hydrogel.

In a preferred embodiment, the composition of the invention comprises R8 and Lucentis at a ratio of at least 40:1, Trehalose at a concentration of at least 200 mg/ml and a hydrogel. In a preferred embodiment, the composition of the invention comprises R8 and Lucentis at a ratio of at least 50:1, Trehalose at a concentration of at least 200 mg/ml and a hydrogel. In a preferred embodiment, the composition of the invention comprises R8 and Lucentis at a ratio of 50:1 to 80:1, Trehalose at a concentration of at least 200 mg/ml and a hydrogel.

In a preferred embodiment, the composition of the invention comprises R8 and Lucentis at a ratio of at least 40:1, Trehalose at a concentration of at least 300 mg/ml and a hydrogel. In a preferred embodiment, the composition of the invention comprises R8 and Lucentis at a ratio of at least 50:1, Trehalose at a concentration of at least 300 mg/ml and a hydrogel. In a preferred embodiment, the composition of the invention comprises R8 and Lucentis at a ratio of 50:1 to 80:1, Trehalose at a concentration of at least 300 mg/ml and a hydrogel.

In a preferred embodiment, the composition of the invention comprises R8 and Lucentis at a ratio of at least 40:1, Trehalose at a concentration of at least 600 mg/ml and a hydrogel. In a preferred embodiment, the composition of the invention comprises R8 and Lucentis at a ratio of at least 200:1, Trehalose at a concentration of at least 600 mg/ml and a hydrogel.

In a preferred embodiment, the composition of the invention comprises R8 and Lucentis at a ratio of at least 40:1, Trehalose and a hydrogel at a concentration of at least 15%-25% w/v. In a preferred embodiment, the composition of the invention comprises R8 and Lucentis at a ratio of at least 40:1, Trehalose and a hydrogel at a concentration of at least 20%-25% w/v.

In a preferred embodiment, the composition of the invention comprises R8 and Lucentis at a ratio of at least 50:1, Trehalose and a hydrogel at a concentration of at least 20%-25% w/v. In a preferred embodiment, the composition of the invention comprises R8 and Lucentis at a ratio of 50:1 to 80:1, Trehalose and a hydrogel at a concentration of at least 20%-25% w/v. In a preferred embodiment, the composition of the invention comprises R8 and Lucentis at a ratio of 50:1 to 80:1, Trehalose at a concentration of at least 100 mg/ml and a hydrogel at a concentration of at least 20%-25% w/v.

In a preferred embodiment, the composition of the invention comprises R8 and Lucentis at a ratio of 50:1 to 80:1, Trehalose at a concentration of at least 200 mg/ml and a hydrogel at a concentration of at least 20%-25% w/v. In a preferred embodiment, the composition of the invention comprises R8 and Lucentis at a ratio of at least 200:1, Trehalose at a concentration of at least 600 mg/ml and a hydrogel at a concentration of at 20%-25% w/v.

An example of a final formulation of the invention comprises 8 mg/ml Lucentis (0.173mM) (10 mg/ml Lucentis, 10mM histidine HCL, 100 mg/ml Trehalose, 0.01% polysorbate 80, pH 5.5), 10 mg/ml R8 (molar ratio R8:Lucentis 50:1), 200 mg/ml Trehalose, 20% w/v Lutrol-F127, 0.01% polysorbate 80, pH 5.5.

In a preferred embodiment, the composition of the invention also comprises a mucoadhesive polymer. Mucoadhesive polymers are already in clinical use to further enhance the delivery of Lucentis to posterior ocular tissues. An example of a suitable mucoadhesive polymer for use in the invention is chitosan. Whilst to date there is no current EU approved medicinal product which includes chitosan, there has been a clinical trial with chitosan, most notably in eye drops for dry eyes: Local Tolerability of Chitosan-N-acetylcysteine Eye Drops in Healthy Young Volunteers ClinicalTrials.gov Identifier: NCT01278784.

An eye drop containing chitosan has been previously available under the trade name Lacrimera by Croma Pharma for the treatment of dry eyes (Schmidl, D. et al., 2017). There is also literature to support the use of chitosan with a good safety and tolerability profile but in limited patient numbers (Fischak C., et al., 2017).

In a preferred embodiment, the composition of the invention comprises a small cationic peptide and a VEGF antagonist at a ratio of 50:1 to 80:1, a hydrogel and a mucoadhesive polymer. In a preferred embodiment, the composition of the invention comprises a small cationic peptide and a VEGF antagonist at a ratio of 50:1 to 80:1, a hydrogel and chitosan. Preferably, the small cationic peptide is R8. Preferably, the VEGF antagonist is Lucentis. Preferably, the small cationic peptide is R8 and the VEGF antagonist is Lucentis. Preferably, the chitosan is present at a concentration of between 0.05% and 0.25% w/v. Preferably the ratio of R8 to Lucentis is 80:1. Preferably the ratio of R8 to Lucentis is 80:1 and the chitosan is present at a concentration of between 0.05% and 0.25% w/v. Preferably the ratio of R8 to Lucentis is 80:1 and chitosan is present at a concentration of 0.1 w/v. Preferably the ratio of R8 to Lucentis is 80:1, the hydrogel is present at a concentration of 20%-25% w/v and chitosan is present at a concentration of 0.1% w/v. Preferably the chitosan is 5k chitosan. Preferably the composition also comprises trehalose.

In a preferred embodiment, the composition of the invention comprises R8 and Lucentis at a ratio of 80:1, a hydrogel and chitosan. In a preferred embodiment, the composition of the invention comprises R8 and Lucentis at a ratio of 80:1, trehalose, a hydrogel and chitosan. In a preferred embodiment, the composition of the invention comprises R8 and Lucentis at a ratio of 80:1, trehalose at a concentration of 10% w/v, a hydrogel at a concentration of 20% w/v and chitosan at a concentration of 0.1% w/v. In a preferred embodiment, the composition of the invention comprises R8 and Lucentis at a ratio of 80:1, trehalose at a concentration of 10% w/v, a hydrogel at a concentration of 20% w/v and 5k chitosan at a concentration of 0.1% w/v.

An example of a final formulation of the invention comprises 10 mg/ml Lucentis, 20 mg/ml R8 (molar ratio R8:Lucentis 80:1), 20% w/v Lutrol-F127, 10% w/v α,α-trehalose dehydrate, 0.1% w/v 5k chitosan, 10 mM histidine HCl, 0.01% w/v polysorbate 80, pH 5.5.

### Therapeutic Indications

The compositions of the present invention can be used to treat or prevent diseases or conditions characterised by VEGF dysfunction. The compositions of the present invention can be used to treat or prevent diseases or conditions where VEGF is functioning aberrantly. The compositions of the present invention can be used to treat diseases currently treated by an VEGF antagonist. The compositions of the present invention can be used to prevent or treat diseases or conditions characterised by VEGF dysfunction such as vasculoproliferative conditions, cancers, oedema or leaky vessels, retinal vein occlusions, diabetic vasculopathy, diseases characterised by retinal angiogenesis, sickle cell disease and retinopathy of prematurity.

The compositions of the present invention can be used to treat or prevent diseases or conditions characterised by VEGF dysfunction such as vasculoproliferative conditions. “Vasculoproliferation”, “vasculoproliferative”, “vasculoproliferative conditions” and similar terms as used herein encompass any and all pathologies related to the aberrant or unwanted development of blood vessels or vascular tissue or cells. For example, both pathogenic angiogenesis (the formation of new blood vessels, for example via new capillary growth from existing blood vessels) and vascular malformation (e.g. telangiectasia, the formation of dilated, tortuous and incompetent vessels, microaneurysms) can be prevented or reduced, as can neovascularisation and vascular endothelial cell proliferation. Also, as is known in the art, neoplastic growth requires the formation of new blood vessels to provide a blood supply to the growing tumour.

Tumours in which vasculoproliferation occurs are therefore also conditions which may be treated, prevented or ameliorated according to the present invention.

Treatment of ocular vasculoproliferative conditions is a preferred embodiment of the invention. Among conditions that can be treated are: age-related macular degeneration (AMD), diabetic retinopathy, retinal vein occlusion, retinopathy of prematurity, macular telangiectasia, or choroidal neovascularisation, diabetic vasculopathy, diseases characterised by retinal angiogenesis and sickle cell disease. Age-related macular degeneration can include “wet” or “dry” AMD.

Treatment of tumours, typically solid tumours, can also be effected, in that preventing angiogenesis in tumours derives the tumour of blood supply. Tumour treatment targets include brain, breast, kidney, colorectal, lung, prostate, head and neck, stomach, pancreatic, skin, cervical, bone, ovarian, testicular and liver tumours.

### Pharmaceutical Compositions, Dosages and Dosage Regimes

Compositions of the invention will typically be formulated into pharmaceutical compositions, together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular, subcutaneous, intraocular or intravitreal administration (e.g., by injection or infusion). Depending on the route of administration, the composition may be coated in a material to protect the composition from the action of acids and other natural conditions that may inactivate the composition.

The pharmaceutical compositions of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be preferable to include isotonic agents, for example, sugars, trehalose, polyalcohols such as mannitol, sorbitol, or sodium chloride in the composition.

Therapeutic compositions typically must be sterile and stable under the conditions of manufacture and storage. The composition can be formulated as a solution, microemulsion, liposome, or other ordered structure suitable to high drug concentration.

When a hydrogel is used a part of the formulation, the composition of the invention is preferably stored at 4° C. before use, owing to gelation at room temperature and pressure.

Pharmaceutical compositions of the invention may comprise additional active ingredients, notably VEGF antagonists as discussed herein.

The compositions of the present invention may be administered for prophylactic and/or therapeutic treatments. In therapeutic applications, compositions are administered to a subject already suffering from a disorder or condition as described above, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”.

In prophylactic applications, compositions are administered to a subject at risk of a disorder or condition as described above, in an amount sufficient to prevent or reduce the subsequent effects of the condition or one or more of its symptoms. An amount adequate to accomplish this is defined as a “prophylactically effective amount”. Effective amounts for each purpose will depend on the severity of the disease or injury as well as the weight and general state of the subject. An example of a condition that may be treated prophylactically in the context of the invention is wet AMD (age-related macular degeneration); one eye may develop the condition before the other, with the first eye being treated once the problem is recognised and the second prophylactically.

A subject for administration of the compositions of the invention may be a human or non-human animal. The term “non-human animal” includes all vertebrates, e.g., mammals and non-mammals, such as non-human primates, sheep, dogs, cats, horses, cows, chickens, amphibians, reptiles, etc. Administration to humans is preferred.

The compositions of the present invention may be administered via one or more routes of administration using one or more of a variety of methods known in the art. As will be appreciated by the skilled artisan, the route and/or mode of administration will vary depending upon the desired results. Preferred routes of administration for compositions of the invention include intravenous, intramuscular, intradermal, intraocular, intraperitoneal, subcutaneous, spinal or other parenteral routes of administration, for example by injection or infusion. The phrase “parenteral administration” as used herein means modes of administration other than enteral and topical administration, usually by injection. Alternatively, the composition of the invention can be administered via a non-parenteral route, such as a topical, epidermal or mucosal route of administration.

In a preferred embodiment of the invention, the composition of the invention is administered topically. The composition can be administered topically to the eye in the form of an eye drop.

A suitable dosage of the composition of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.1 μg/kg to about 100 mg/kg body weight of the patient to be treated. For example, a suitable dosage may be from about 1 μg/kg to about 10 mg/kg body weight per day or from about 10 g/kg to about 5 mg/kg body weight per day. For intraocular administration, a suitable dosage may be from about 1 μg-1 mg.

Topical formulations may contain 10 mg/mL Lucentis with 12.5 mg/mL R8. A typical eye drop volume may be 0.05 mL, therefore a typical dose may be; one drop per day: 50 μg Lucentis with 62.5 μg R8, two drops per day: 100 μg Lucentis with 125 μg R8, or four drops per day: 200 μg Lucentis with 250 μg R8. Dosage regimens may be, for example, every 12 hours or 24 hours. Dosage regimens may be, for example, once daily or twice daily.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Administration may be in single or multiple doses. Multiple doses may be administered via the same or different routes and to the same or different locations. Alternatively, doses can be via a sustained release formulation, in which case less frequent administration is required. Dosage and frequency may vary depending on the half-life of the antagonist in the patient and the duration of treatment desired.

The composition of the invention can comprise more than one VEGF antagonist as described herein. The composition of the invention can be administered in combination with other therapies for the treatment of the conditions described herein.

Also within the scope of the present invention are kits comprising compositions of the invention. The kits may contain instructions for use. The kit may further contain one or more additional reagents, such as an additional therapeutic or prophylactic agent as discussed above.

The invention encompasses a method of prevention or treatment of a condition characterised by VEGF dysfunction, said method comprising administering a prophylactically or therapeutically effective amount of the composition as defined herein to a patient in need thereof.

The invention encompasses a method of prevention or treatment of a condition characterised by VEGF dysfunction as described herein, wherein the condition characterised by VEGF dysfunction is cancer, oedema or leaky vessels.

The invention encompasses a method of prevention or treatment of a condition characterised by VEGF dysfunction as described herein, wherein the condition characterised by VEGF dysfunction is a vasculoproliferative condition.

The invention encompasses a method of prevention or treatment of a vasculoproliferative condition as described herein, wherein the vasculoproliferative condition comprises neovascularisation, vascular endothelial cell proliferation, angiogenesis, telangiectasia or microaneurysms.

The invention encompasses a method of prevention or treatment of a vasculoproliferative condition of the eye, said method comprising administering a prophylactically or therapeutically effective amount of the composition as defined herein to a patient in need thereof.

The invention encompasses a method of prevention or treatment of a condition characterised by VEGF dysfunction as described herein, wherein the composition is formulated for topical administration.

The invention encompasses a method of prevention or treatment of a condition characterised by VEGF dysfunction as described herein, wherein the composition is formulated for topical administration to the eye.

The invention encompasses a method of prevention or treatment of a condition characterised by VEGF dysfunction as described herein, wherein the composition is administered to the eye by eye drop.

The invention encompasses the composition of the invention as defined herein, for use in the prevention or treatment of a condition characterised by VEGF dysfunction.

The invention encompasses the composition of the invention as defined herein, for use in the prevention or treatment of a condition characterised by VEGF dysfunction, wherein the condition characterised by VEGF dysfunction is cancer, oedema or leaky vessels.

The invention encompasses the composition of the invention as defined herein, for use in the prevention or treatment of a condition characterised by VEGF dysfunction, wherein the condition characterised by VEGF dysfunction is a vasculoproliferative condition.

The invention encompasses the composition of the invention as defined herein, for use in the prevention or treatment of a condition characterised by VEGF dysfunction, wherein the vasculoproliferative condition comprises neovascularisation, vascular endothelial cell proliferation, angiogenesis, telangiectasia or microaneurysms.

The invention encompasses the composition of the invention as defined herein, for use in the prevention or treatment of a vasculoproliferative condition of the eye.

The invention encompasses the composition of the invention as defined herein, for use in the prevention or treatment of a condition characterised by VEGF dysfunction, wherein the composition is formulated for topical administration.

The invention encompasses the composition of the invention as defined herein, for use in the prevention or treatment of a condition characterised by VEGF dysfunction, wherein the composition is formulated for topical administration to the eye.

The invention encompasses the composition of the invention as defined herein, for use in the prevention or treatment of a condition characterised by VEGF dysfunction, wherein the composition wherein the composition is administered to the eye by eye drop.

The invention encompasses a pharmaceutical agent for the prevention or treatment of a condition characterised by VEGF dysfunction, comprising a composition comprising a small cationic peptide and a VEGF antagonist as an active ingredient.

The invention encompasses the use of a composition comprising small cationic peptide and a VEGF antagonist in the manufacture of a pharmaceutical composition for the prevention or treatment of a condition characterised by VEGF dysfunction.

The present invention encompasses a method of treating a tumour that exhibits vasculoproliferation, said method comprising administering a prophylactically or therapeutically effective amount of the composition as defined herein to a patient in need thereof.

The present invention encompasses a method of treating a tumour that exhibits vasculoproliferation as described herein, wherein the tumour is selected from brain tumour, breast tumour, kidney tumour, colorectal tumour, lung tumour, prostate tumour, head and neck tumours, stomach tumour, pancreatic tumour, skin tumour, cervical tumour, bone tumour, ovarian tumour, testicular tumour and liver tumours.

The invention encompasses the composition described herein for use in the treatment of a tumour the exhibits vasculoproliferation.

The following Examples illustrate the invention.

### EXAMPLES

We have previously established that Annexin A5 (AnxA5) functionalised liposomal nanoparticles can deliver large protein molecules including antibodies across the cornea to the back of the eye on topical application by promoting transcytosis. During scale-up of the AnxA5/Lucentis nanoparticles, we encountered two issues:

i) the duration of the electrostatic AnxA5/nanoparticle interaction is short-lived

ii) Lucentis interacts with nanoparticles at these concentrations causing aggregation

which reduced effective shelf-life. Although direct covalent conjugation of AnxA5 to Lucentis loaded nanoparticles was technically feasible, it was extremely difficult and prohibitively expensive to manufacture.

Human AnxA5 contains a total of 19 arginine residues. All annexins from vertebrates contain an invariant arginine (R) in each domain. AnxA5 has a four-repeat domain structure which in turn forms higher order intermolecular structures (AnxA5 dimers/trimers) on interacting with cell membranes containing PS or PE. Although not adjacent in the primary amino acid sequence, on folding (secondary and tertiary structure), AnxA5 R residues are brought into close proximity and interact with each other via formation of intramolecular and intermolecular interactions which are key to the function of the annexin V protein.

8 of the 19 arginine residues have active membrane-binding activities. R43, R115, R199 and R274 are highly conserved arginine residues in the AnxA5 sequence, and shown to have differential effects on the PS binding ability, tertiary structure, and proteolytic susceptibility of AnxA5. R23E and R61E mutations have a significant reduction in adsorption to phospholipid membranes relative to the wildtype protein. R23 found to be major determinant for interfacial phospholipid binding and participates in an intermolecular salt bridge that is key for trimer formation on the membrane surface. R163 plays a central role in the selective binding of AnxA5 to Protein Kinase C and mutation of this residue abolishes this interaction.

AnxA5 has eight calcium binding sites involved in binding to cells and vesicles via lipid interaction.

Arginine rich peptides can be used as cell-penetrating peptides through promotion of intracellular uptake or endocytosis. This has recently been suggested to occur due through an interaction with PS and PE promoting negative membrane curvature in PS/PE containing membranes. A similar mechanism of action is proposed for AnxA5 mediated endocytosis.

Based on the above, we sought to determine whether the annexin-derived sequence RRRRRRRR (R8) (SEQ ID NO: 1) could be used as a replacement for AnxA5 initially in R8 coupled liposomes, to enhance transcytosis of encapsulated anti-VEGF across corneal epithelial barriers.

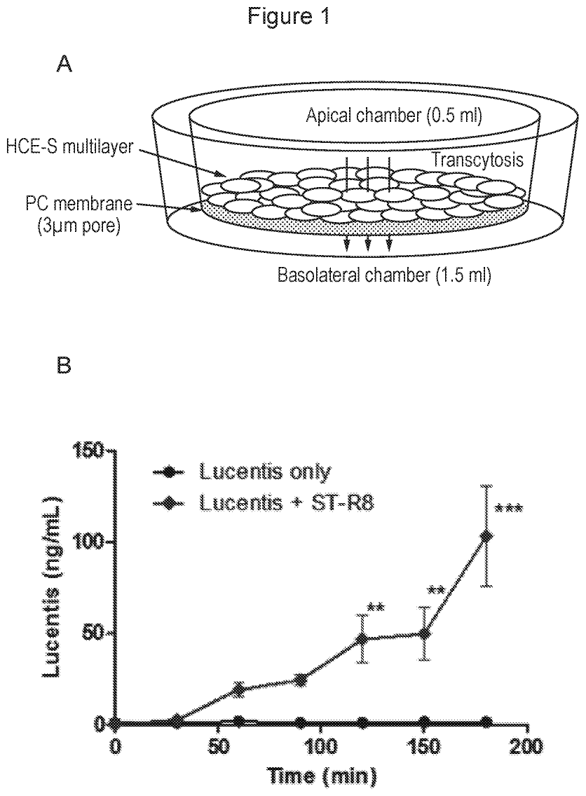

Using a previously described in vitro HCE-S transcytosis assay (illustration of the in vitro human corneal epithelial (HCE-S) barrier model used in this study shown in FIG. 1), we found that the presence of ST-R8 significantly enhanced the transcytosis of anti-VEGF (Lucentis) loaded liposomes. R8 alone without liposomes was investigated as a control. Surprisingly, this formulation also showed a dramatic enhancement in transcytosis.

### Materials and Methods

### In Vitro HCE-S Assays

HCE-S cells were cultured as a monolayer in 90% DMEM supplemented with 10% heat inactivated FBS and penicillin-streptomycin (100 U/mL). Cells were cultured at 37° C. in a humidified incubator with 5% CO₂ and growth medium was replaced every three days. HCE-S populations were grown to 80% confluence before passage to 20% using 0.25% Trypsin-EDTA. Prior to each experiment cell populations were estimated using a haemocytometer and trypan blue exclusion assay.

### Viability Assay

HCE-S monolayers for uptake assays were prepared by seeding 96 well plates with 1×10⁴ cells/well and maintaining for 5 days replacing the medium every other day. After this time, medium was replaced with that containing 100 μM calcein loaded PLVs in the presence or absence of 200 nM AnxA5. Cells were incubated with PLVs at 37° C. for 3 h protected from light. Epithelial monolayers were then washed three times in ice cold PBS (with calcium and magnesium) to remove free PLVs, followed by a single wash in ice cold isosmotic citrate buffer (pH 3) to remove surface bound PLVs before applying a PBS wash containing 1% Triton X-100 to solubilise membranes. Upon completion of each washing step, calcein fluorescence was measured using a 485 nm excitation and 515 nm emission with a SAFIRE micro plate reader (TECAN) HCE-S monolayers for viability assays were prepared by seeding 96 well plates with 1×10⁴ cells/well and maintaining for 24 h at 37° C. in a humidified incubator with 5% CO₂. After this time, medium was replaced with that containing specified concentrations of anti-VEGF (Lucentis or Avastin) in the presence or absence of varying concentrations of R8 or ST-R8 (0-200 molar ratio). Cells were incubated with anti-VEGF/R8 solutions for 24 h before viability of HCE-S cells in response to was assessed using the AlamarBlue (Invitrogen) cell viability assay according to manufacturers instructions. Briefly, 10 μl AlamarBlue solution was added to each well-plate and incubated for 2.5 h after which time the intensity of AlamarBlue fluorescence (Excitation 570 nm/Emission 585 nm) was recorded from which percentage cell viability was determined using equation 1;

$$Viability ( \% ) = 100 ( \frac{( T - N )}{( H - N )} )$$

where T is the fluorescence intensity of the test well, H is the fluorescence intensity of a population of untreated (healthy) cells and N denotes a population of cells treated with a highly cytotoxic insult (0.1% Triton-X100).

### Transwell Barrier Model Establishment

HCE-S barrier models for transcytosis assays were prepared using an adaptation of a previously described method.^{[1]} HCE-S cells (5×10⁴ cells/well) were seeded on transwell inserts (poly-carbonate, 3 μm pore size, 1.13 cm²) and maintained for 7 days refreshing the medium in the apical and basal chambers every second day. After this time cells were cultured for a further 10 days at an apical air interface to encourage development of a multilayer barrier. Prior to each experiment culture medium was replaced with phenol red free DMEM and barrier integrity measured via transepithelial resistance (TER). TER values were obtained in the range 300-600 Ωcm² and only epithelial barriers with TERs>300 Ω·cm², the accepted threshold for tight epithelial barriers, were used for transcytosis experiments.

### Anti-VEGF Transcytosis Experiments

Permeation studies were conducted to determine the rate of anti-VEGF transcytosis in the presence/absence of varying concentrations of R8. Experiments were initiated by adding 1.5 ml serum- and phenol red-free DMEM to the basal chamber and 0.5 ml to the apical chamber containing 0.1 mg/mL Avastin or Lucentis in the presence of varying molar ratios of R8. After 3 h, 100 μl of medium was withdrawn from the basal chamber and the concentration of anti-VEGF in the basal chamber was determined using established and commercially available sandwich ELISA techniques (KPL Protein Detector HRP Microwell Kit, Anti-Human Cat #54-62-10 with Jackson Immuno Research [309-005-082] AffiniPure Rabbit Anti-Human IgG capture antibody).^{[1]} The presence of R8 was found to not significantly impact anti-VEGF detection using this ELISA technique. The permeability coefficient (Pₐₚₚ) of each anti-VEGF was determined as previously described^{[1}] using equation 1;

$${P}_{app} = ( \frac{( {C}_{R} / {C}_{D} )}{{t}_{min}} ) * ( \frac{{V}_{R}}{( A * 60 )} )$$

where C_{R} is the final concentration of anti-VEGF recovered from the ELISA assay, C_{D} is the initial concentration of anti-VEGFs added to the assay, tₘᵢₙ is the transcytosis time (180 min), V_{R} is the volume of the receiving chamber (1.5 ml) and A represents the filter area (1.12 cm²).

### Stability of R8 Containing Formulations

Lucentis (0.1 mg/mL) was added to each well of a 96 well plate in the presence of varying concentrations of R8 in the same buffer (10 mM Histidine HCL, 10% (w/v) Trehalose and 0.1% (v/v) polysorbate 80) and additional trehalose was added (up to a final concentration of 600 mg/mL). Samples were incubated at 25° C. and aggregation monitored by recording absorbance at 600 nm over time using a TECAN Safire plate-reader.

Anti-VEGF activity was recorded using a VEGF binding ELISA. Briefly, a Nunc Maxisorp (cat 442404) high affinity, protein binding ELISA plate was coated in 0.05 mL of 0.25 μg/mL human VEGF₁₆₅ Peprotech [Cat #100-20] dissolved in PBS buffer overnight at 4° C. Plates were washed (three times, five minutes each) with Tris-buffered saline containing 0.005% Tween-20 before blocking for 1.5 h with Protein free (TBS), Thermo scientific (Cat #37570). Plate was washed before samples and standards of known anti-VEGF concentrations were added and the plate incubated for a further 2 h. After this time the plate was washed before 0.05 mL/well of 0.1 mg/mL of detection antibody was applied (Affinity purified Goat anti-Human IgG Peroxidase labelled (KPL, 04-10-06)) for a further 1 h. Finally, the plate was washed before developing with BM Blue POD substrate, soluble (Sigma, 000000011484281001) for 10 minutes before stopping the reaction with 1M Hydrochloric acid and recording absorbance at 450 nm against a reference wavelength of 690nm. Results are presented as the ratio of IC₅₀ of each formulation over the IC₅₀ of commercially available anti-VEGF formulations.

### In Vivo Rabbit Studies

All animal experiments were performed in compliance with the ARVO Statement for the Use of Animals in Ophthalmic and Vision Research. New Zealand rabbits received unilateral topical instillations of 0.03 mL anti-VEGF in the presence of R8 (varying molar concentrations) either as a liquid or hydrogel formulations without anaesthesia. At the specified time after application animals were euthanized before enucleation, washing (PBS) and snap-freezing and dissection of ocular tissues. Samples were weighed and cryohomogenised before the concentration of anti-VEGF per gram of eye tissue determined using established sandwich ELISA protocols.

Corneal toxicity was assessed in vivo in rabbit eyes, where evidence of redness, swelling, discharge, ulceration, haemorrhaging or scarring in the treated eye was assessed. Epithelial toxicity was investigated using fluorescein staining, using standard protocols.

### Reference

[1] B. Davis, E. Normando, L. Guo, P. O'Shea, S. Moss, S. Somavarapu, M. Cordeiro, Small 2014, 10, 1575.

### Example 1—R8/Anti-VEGF Molar Ratios and Transcytosis

Initial formulations concerned replacing AnxA5 with R8 in liposomal and micelle formulations, but we found that electrostatic interactions between R8 and protein drugs also had a beneficial effect for drug delivery. A schematic of the in vitro HCE-S assay used to measure transport across corneal barriers in vitro is shown in FIG. 1A. FIG. 1B indicates that presence of R8 (Steryl-R8) enhances the transport of Lucentis across corneal barriers in vitro. Lucentis (+/−Steryl-R8) were applied to the Apical chamber and the concentration crossing the barrier was determined from the basolateral chamber at different timepoints using a Lucentis sandwich ELISA. Intact HCE-S barrier was confirmed by measuring transepithelial resistance (>600 Ω·m²) before each assay.

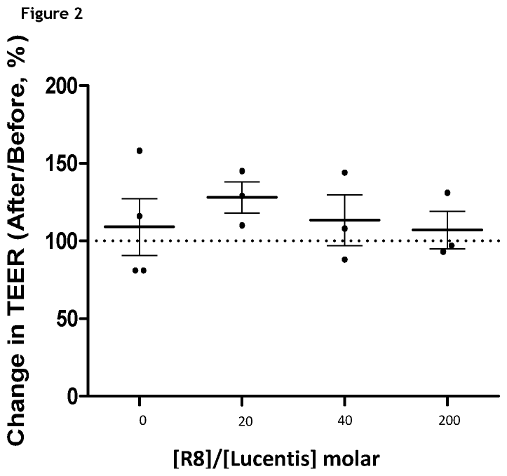

R8 enhanced transcytosis across cornea via a transcellular mechanism without affecting cell viability or disruption of corneal barrier integrity (FIG. 2). Addition of R8 and Lucentis to HCE-S transwells for 3 h induced no significant change in transepithelial resistance suggestive of a transcellular delivery mechanism. This is contrary to what has been reported with poly-arginine (36 kDa) polypeptides for which the mechanism has been suggested to be reversible disruption of tight junctions (TJs) (Nemoto et al. Biol Pharm Bull. 2007 September; 30(9):1768-72.)

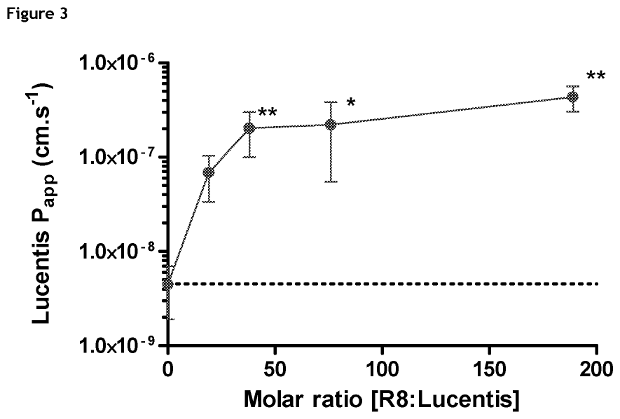

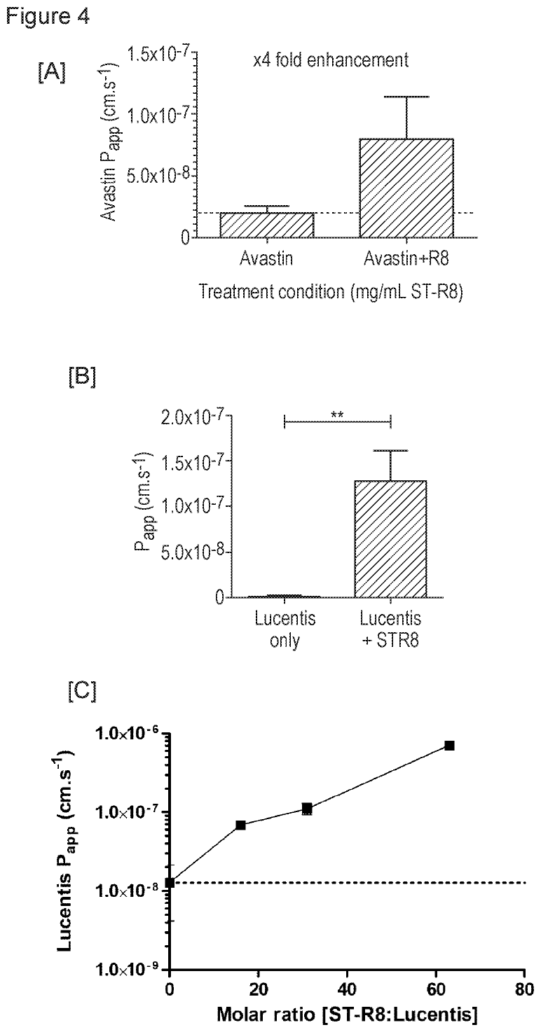

R8 enhanced delivery across corneal barriers in vitro in a concentration dependent manner. A surprising finding in our in vitro studies was that there is evidence that R8 promoted delivery across corneal cells. Another method to monitor the extent of delivery across corneal barrier models is to calculate the drug permeability coefficient (Pₐₚₚ) as described in Davis et al 2014. FIG. 3 shows that R8 enhanced the delivery of the anti-VEGF Lucentis across the HCE-S barrier in a concentration dependent manner. Molar ratios of R8:Lucentis of 40:1 or more significantly enhanced corneal transcytosis in vitro. FIG. 4 shows that R8 enhances the delivery of the both VEGF antagonists Avastin and Lucentis across the HCE-S barrier. In addition, Stearyl-R8 enhanced the delivery of the anti-VEGF Lucentis across the HCE-S barrier in a concentration dependent manner.

### Example 2—Toxicity and Stability Testing

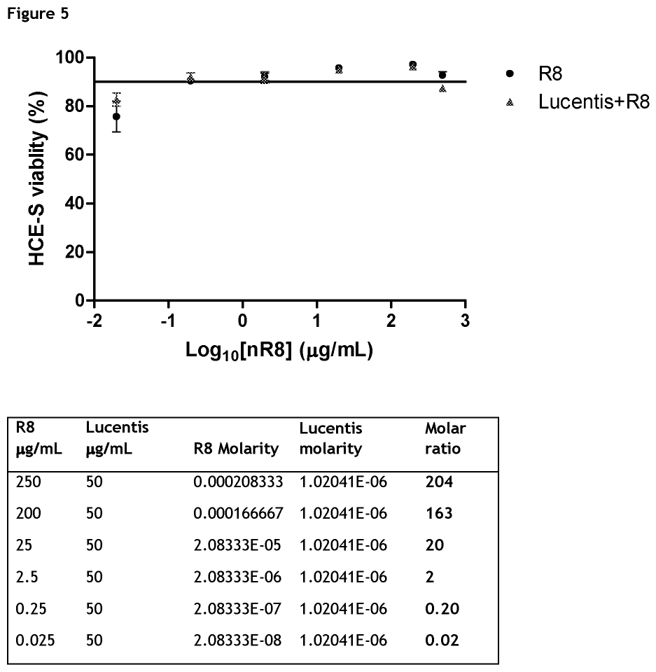

Using the alamarBlue cell viability assay R8 was found to be non-toxic to in vitro human corneal cell cultures (HCE-S) after 24 h exposure. (FIG. 5). R8 was found to be well tolerated in the absence or presence of Lucentis after 24 h exposure. R8 caused no significant reduction in cell viability at the highest concentrations assessed (200/1 molar ratio).

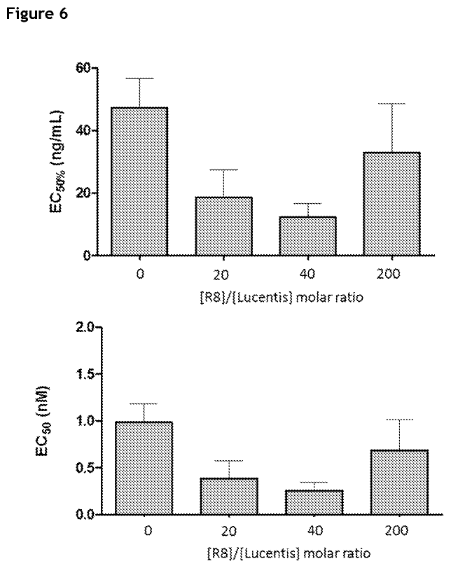

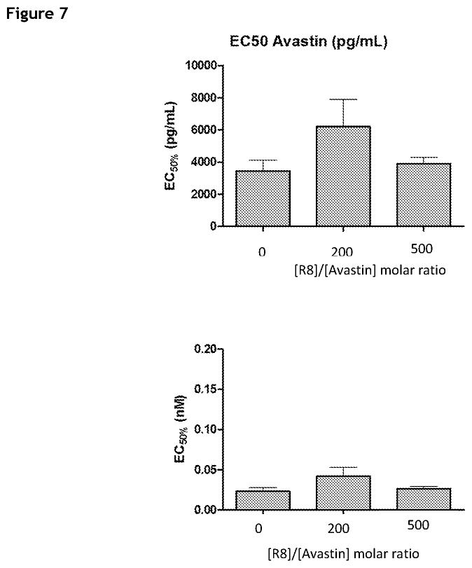

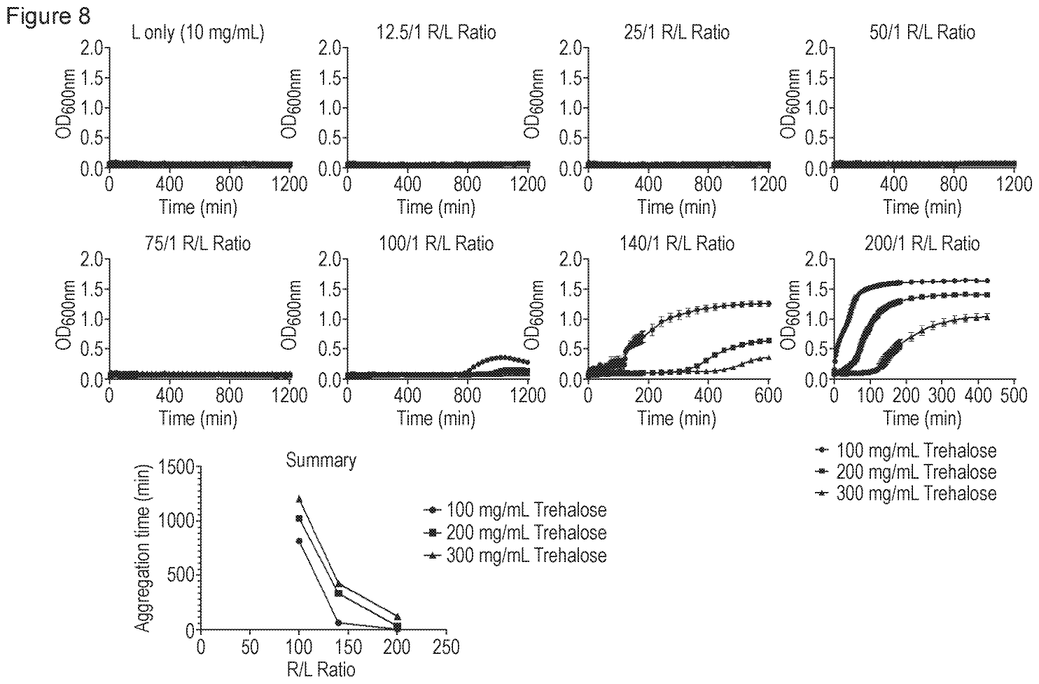

The presence of R8 did not negatively affect the VEGF binding activity of Lucentis. (FIG. 6). The presence of R8 did not impact the Lucentis sandwich ELISA used in rabbit studies. Using a hVEGF-165 ELISA to assess Lucentis activity, the addition of R8 in a molar ratio of 200/1 of R8/Lucentis caused no significant impediment in Lucentis binding to hVEGF. In fact the data suggested that the presence of these peptides may mildly increase the affinity of Lucentis for hVEGF. Similar results were achieved using Avastin (FIG. 7).

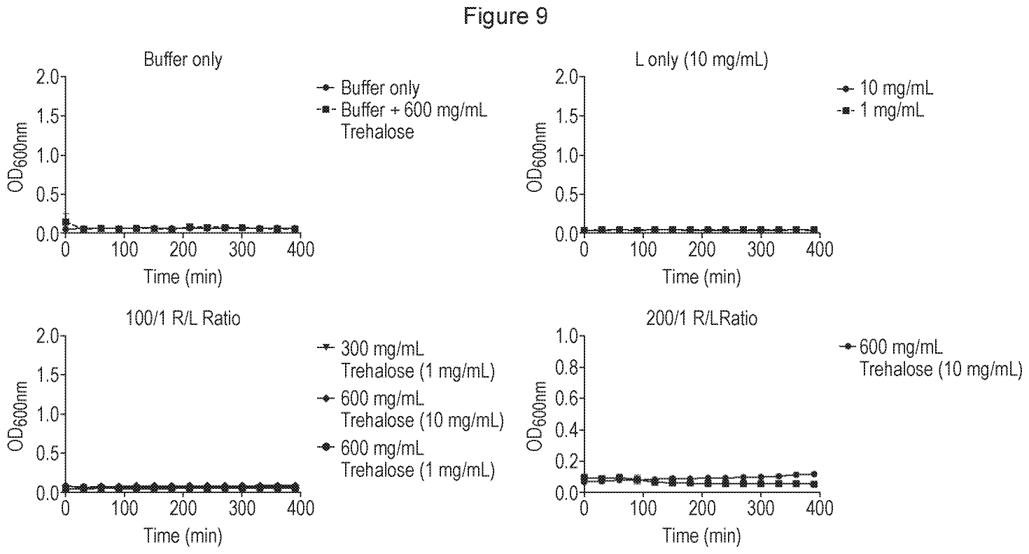

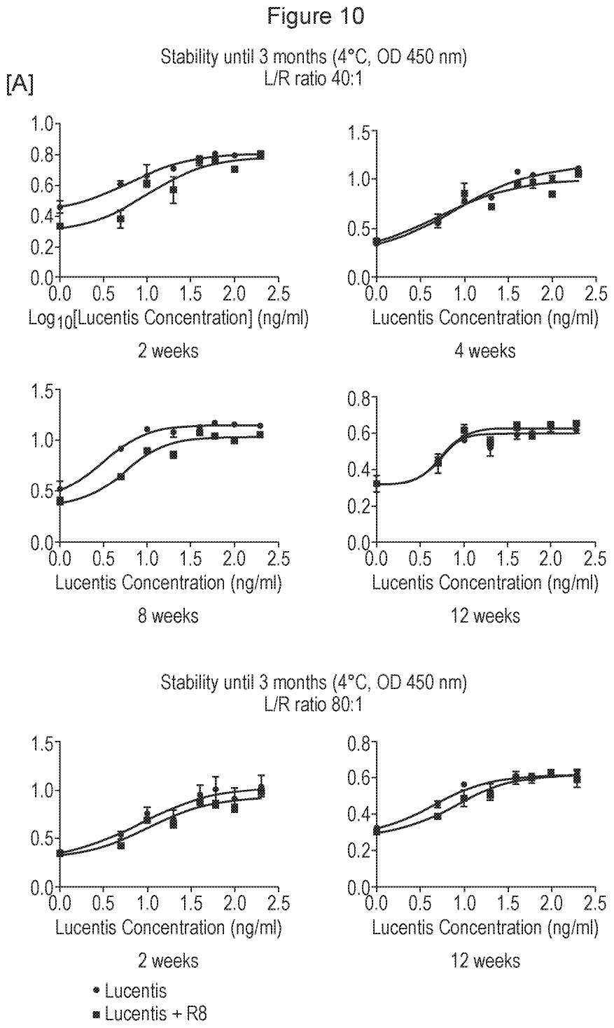

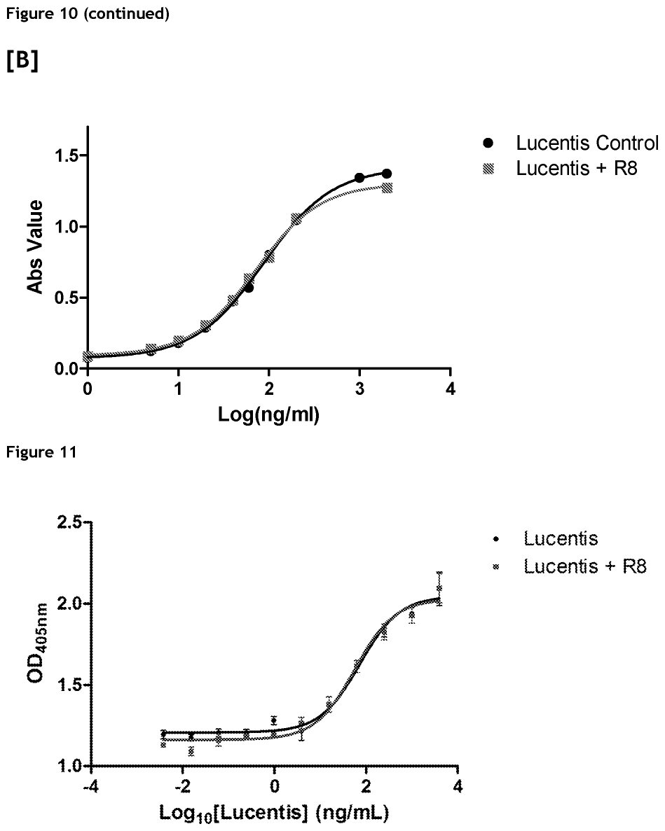

The stability of the R8:Lucentis formulations were investigated by measuring aggregation over time using 600 nm absorbance at 25° C. (FIG. 8). Ratios of 50 to 100:1 R/L, were the most stable. However, aggregation was delayed by increasing the Trehalose concentration in the formulation. The results suggest a ratio of 50:1 would be suitable the formulation of the invention. Similar results were seen by reducing Lucentis concentration while maintaining the R/L, ratio, or by increasing the Trehalose concentration further, to 600 mg/mL (FIG. 9). R8/Lucentis combinations at ratios of 40:1 and 80:1 were found to be stable for 3 months at 4° C. (FIG. 10A). An R8/Lucentis combination at a ratio of 50:1 was also found to be stable for 3 months (12 weeks) at 4° C. (FIG. 10B).

In addition, the presence of R8 did not interfere with the anti-VEGF detection ELISA (FIG. 11).

### Example 3—In Vivo Studies

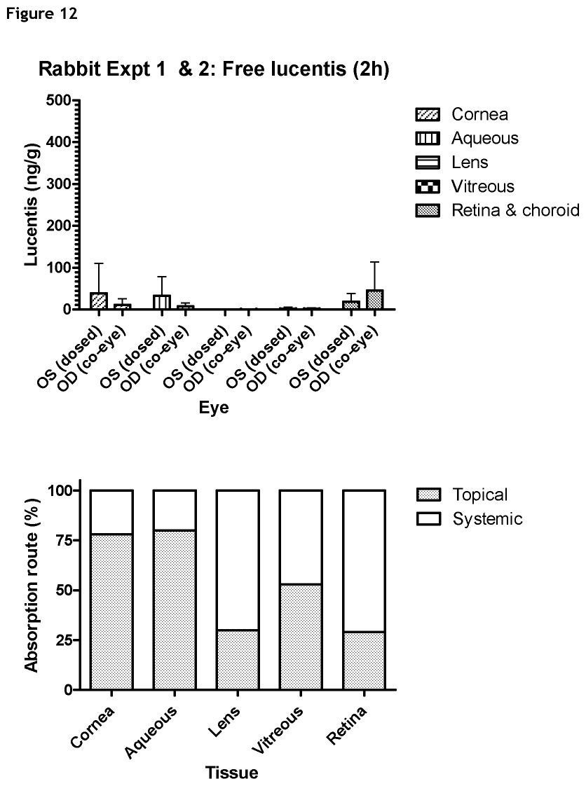

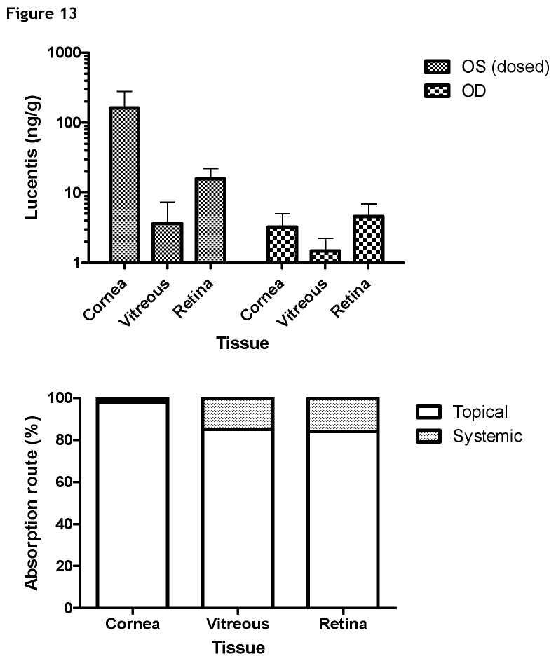

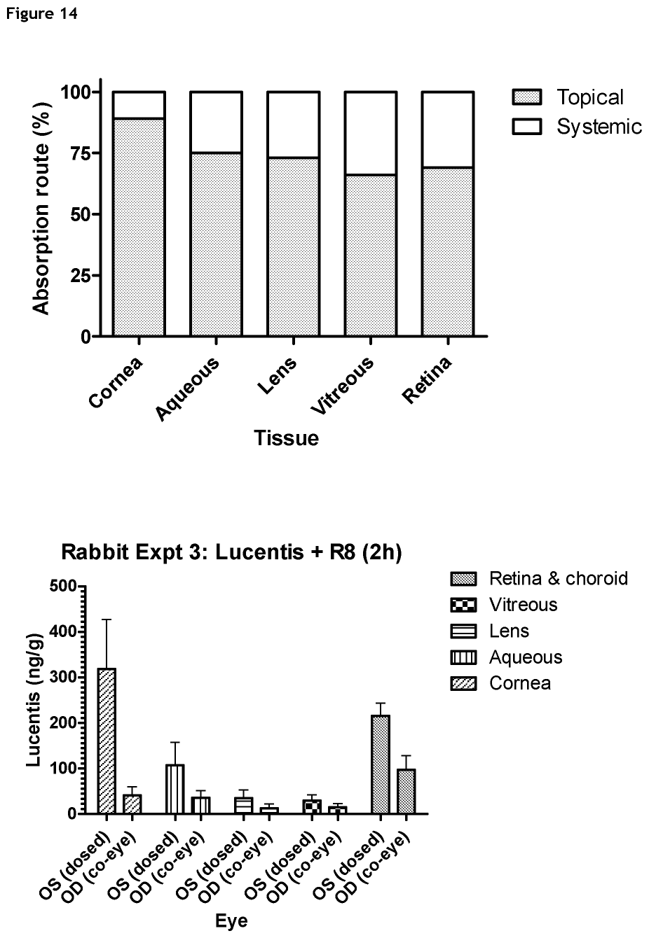

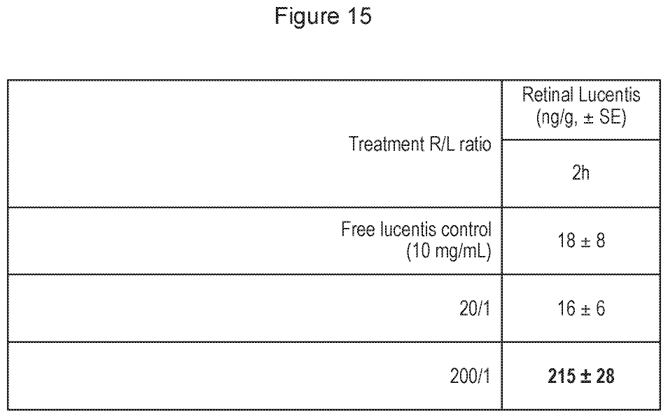

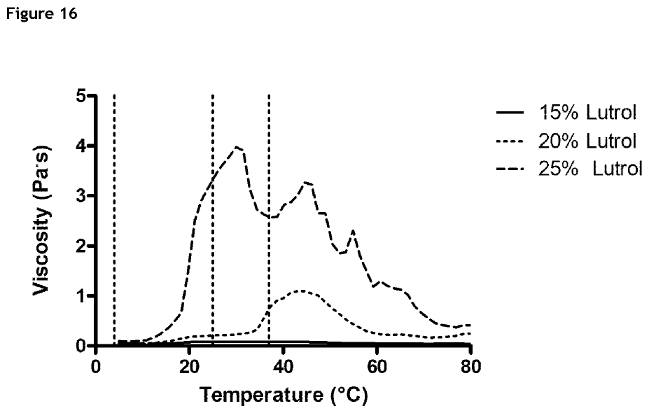

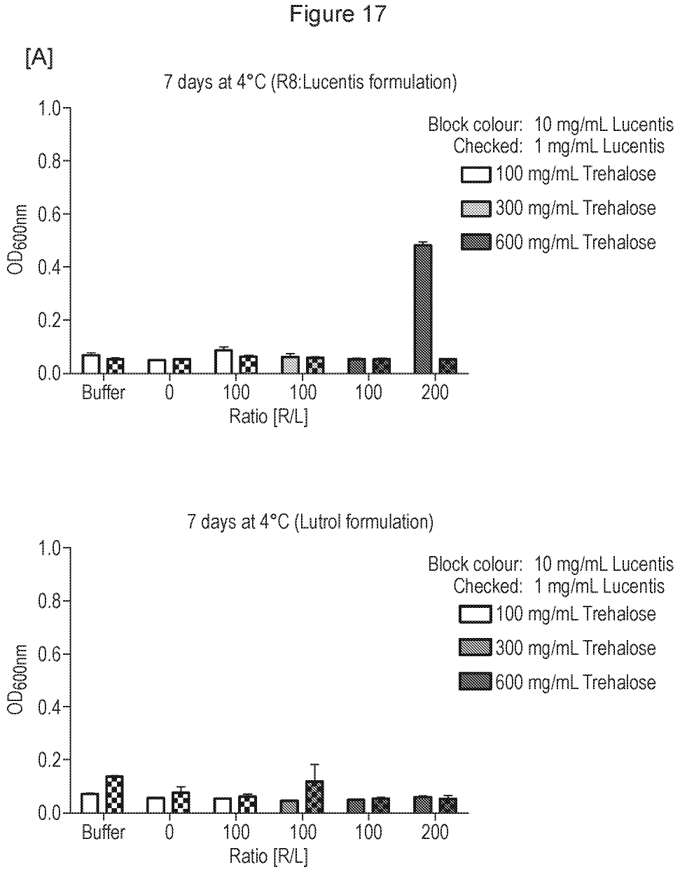

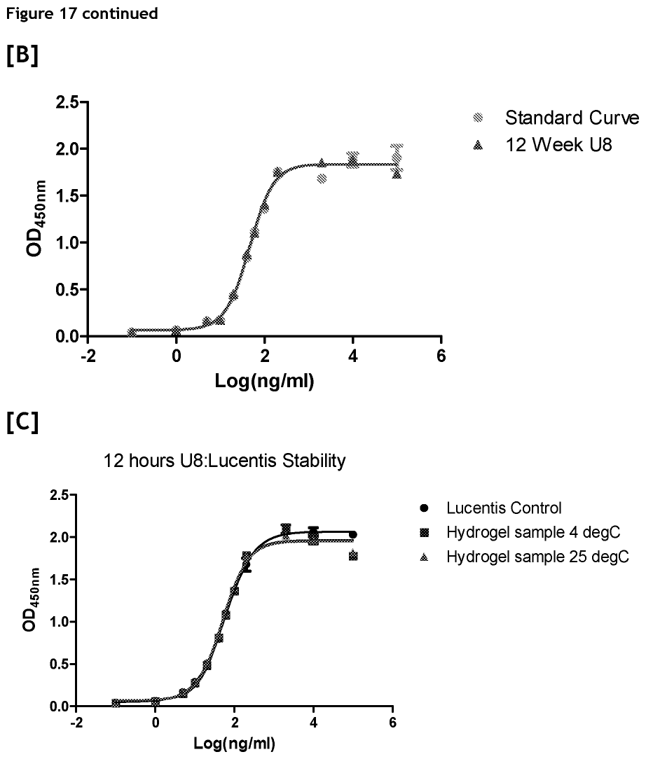

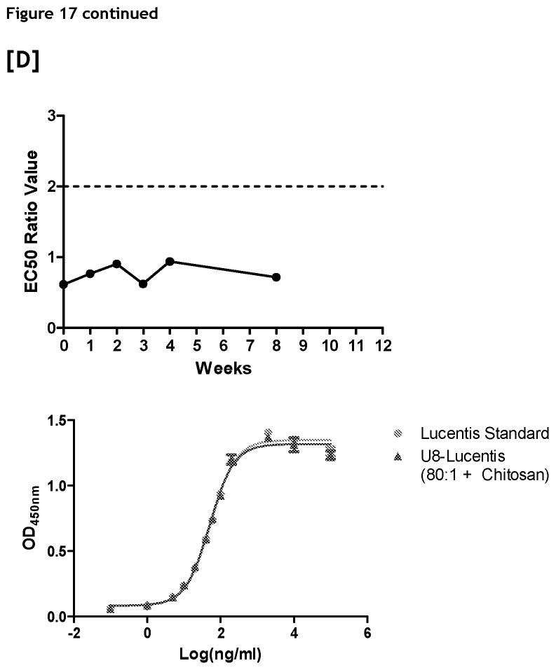

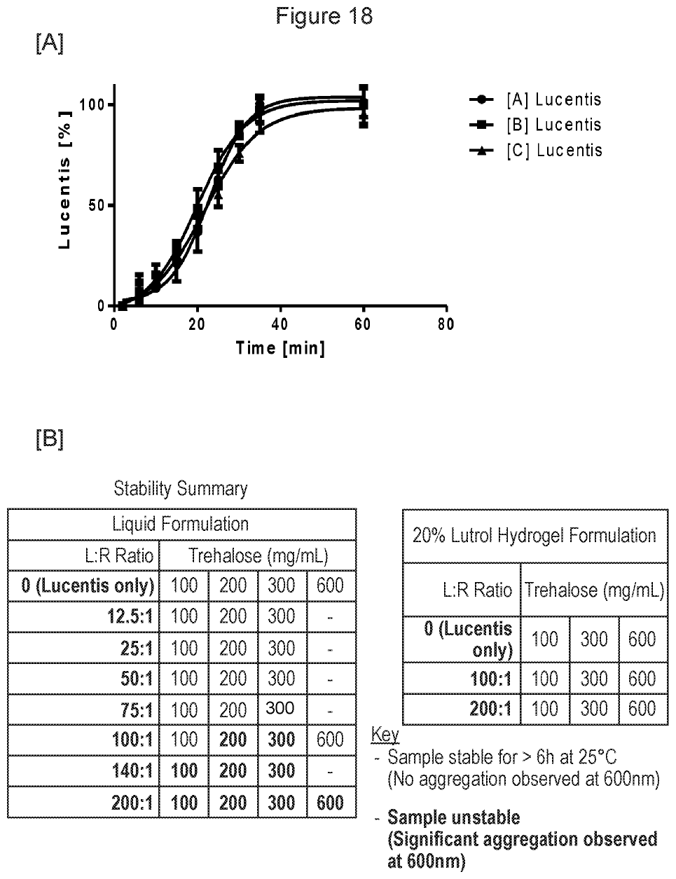

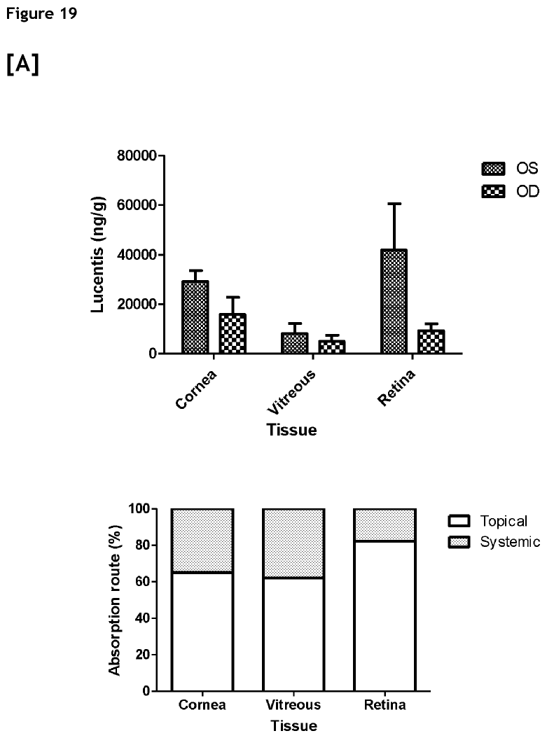

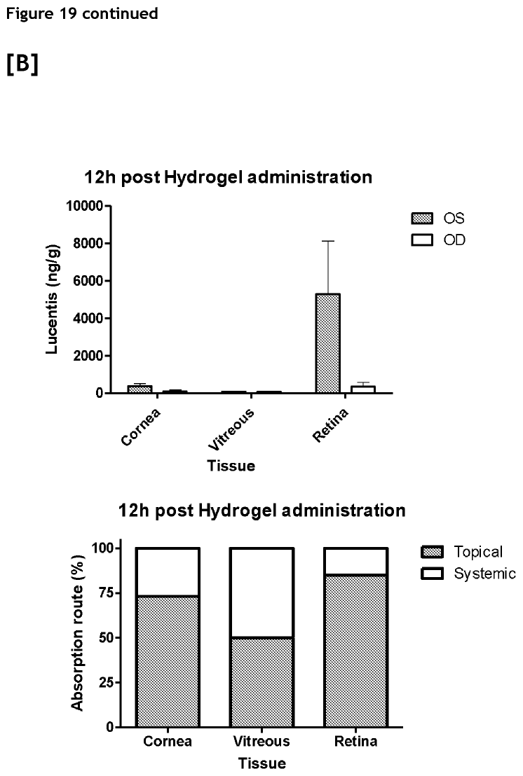

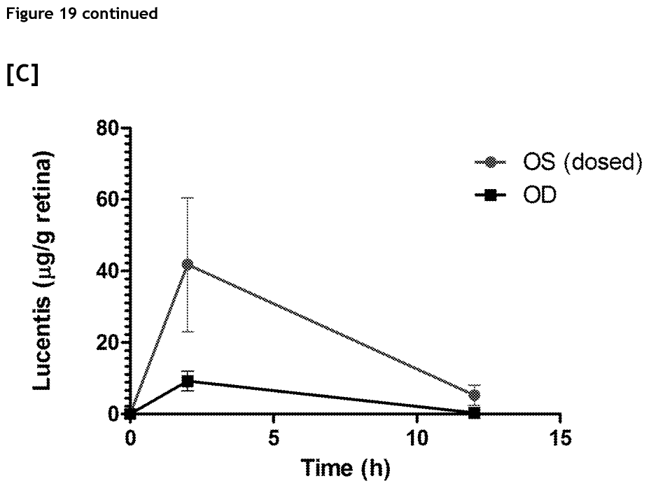

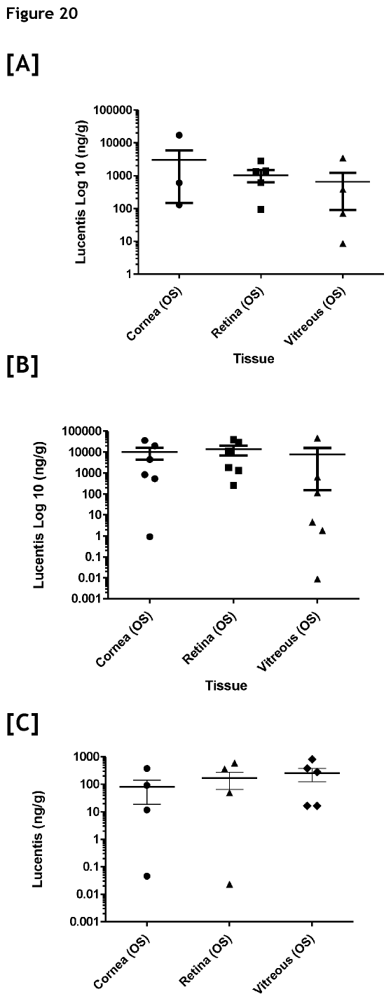

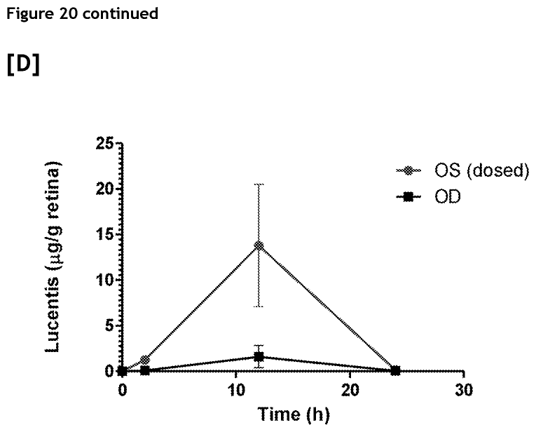

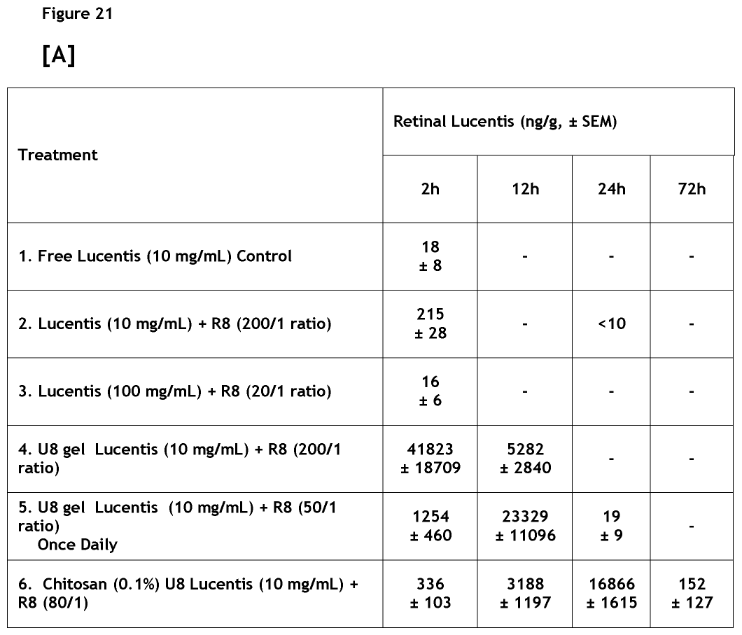

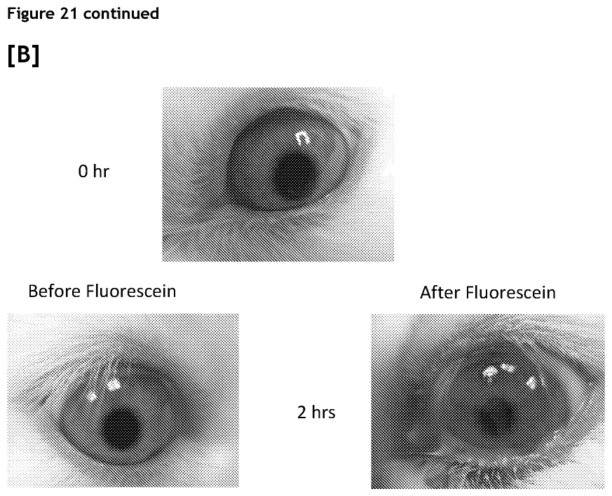

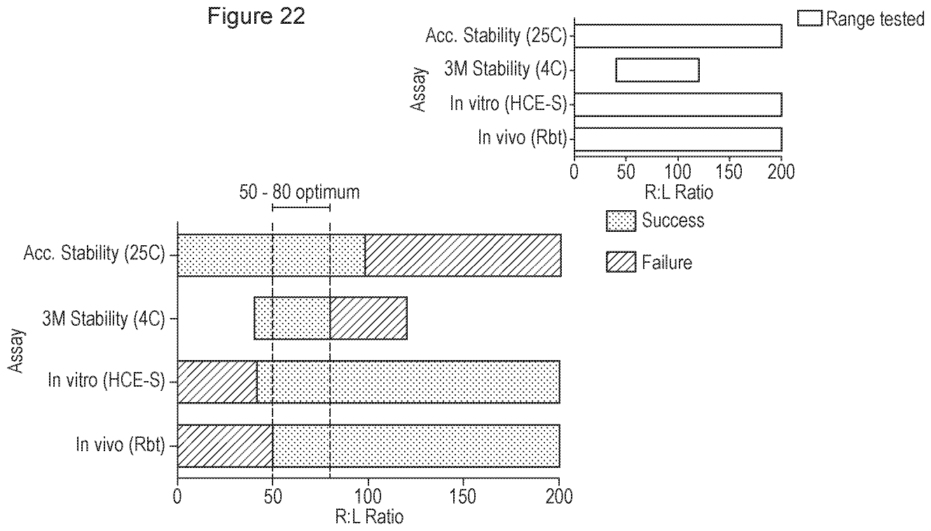

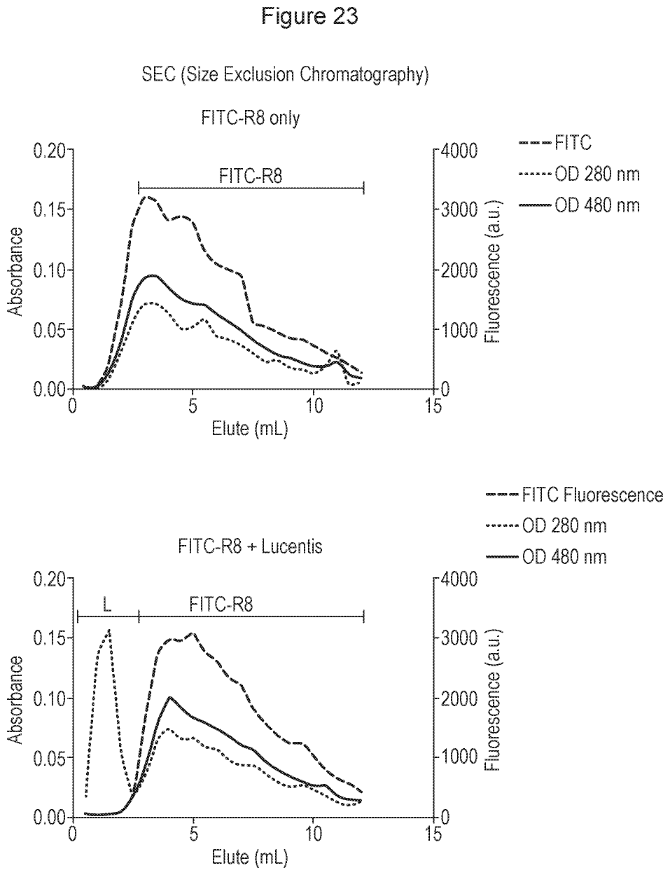

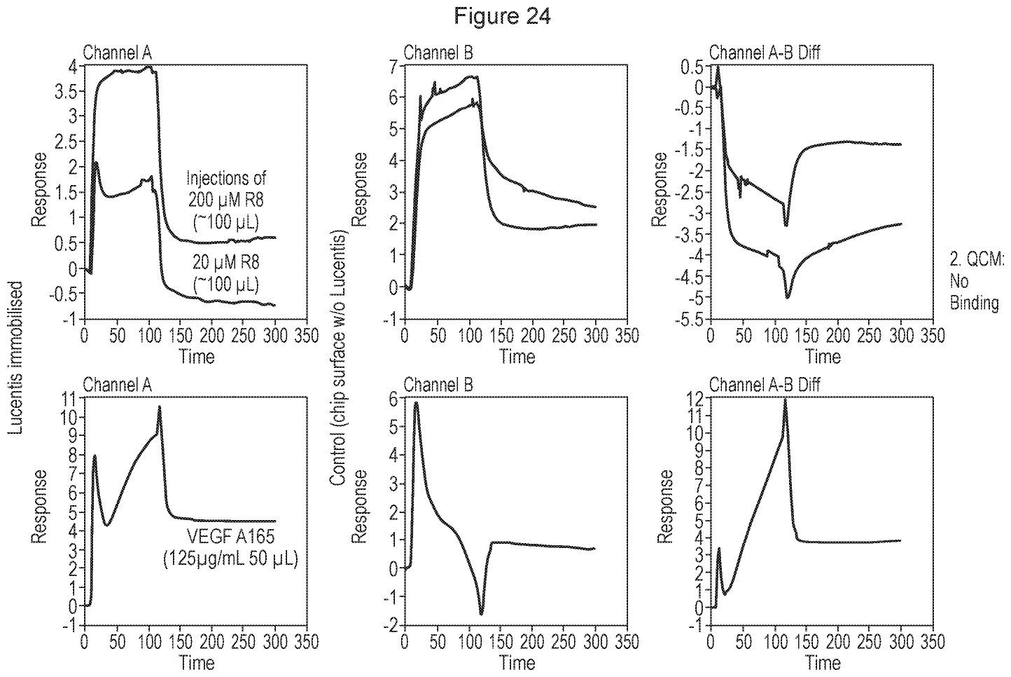

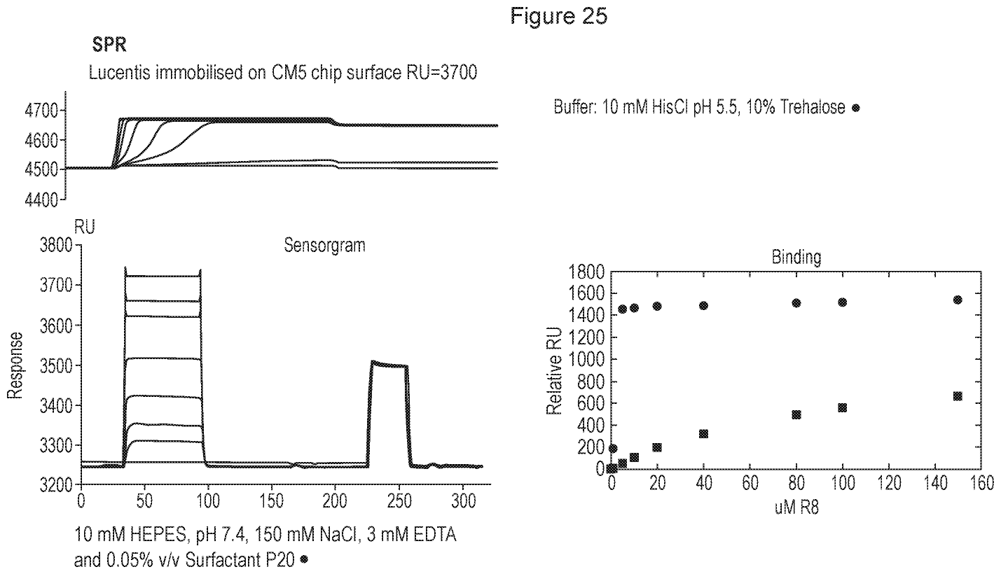

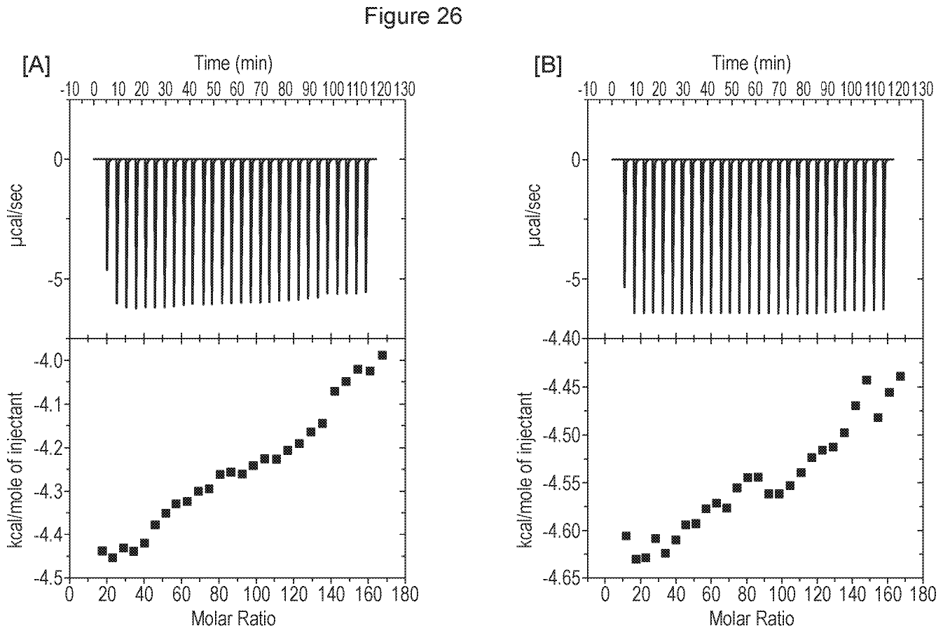

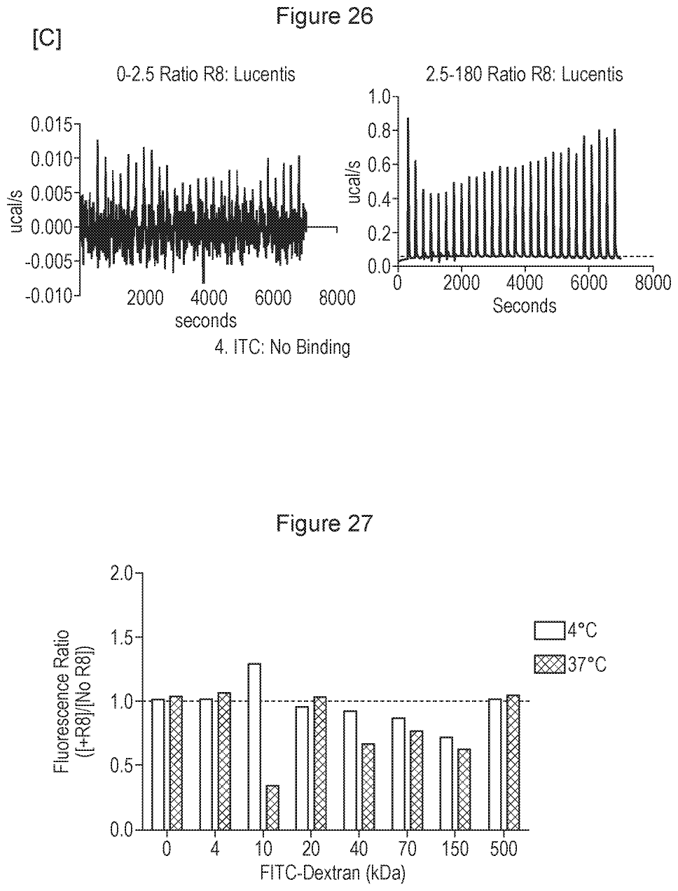

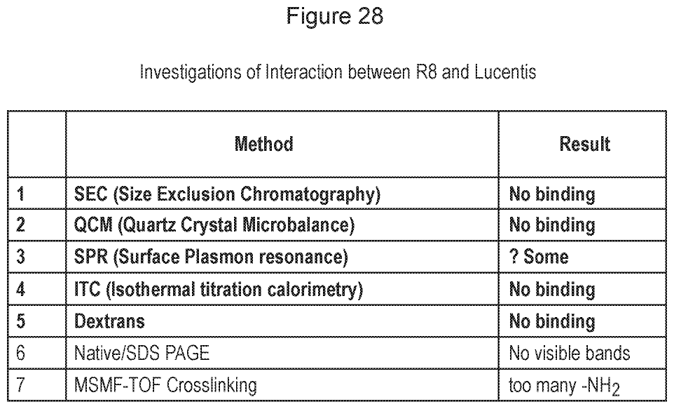

A control in vivo experiment to determine the proportion of free Lucentis reaching the retina is shown in FIG. 12. The proportion of dosed free Lucentis reaching the retina by the topical route was ˜30%. Furthermore, R8:Lucentis at a ratio of 20/1 did not enhance delivery of Lucentis to the retina after 2 h. A 100 mg/mL Lucentis with 50 mg/mL R8 combination provided no significant improvement in Lucentis delivery to the retina compared to Lucentis only control (n=3) (16+/−6 ng/g versus 18+/−8 ng/g). (FIG. 13). This suggests that the R8:Lucentis ratio is important. This ratio of R peptide to anti-VEGF is similar to that reported in the de Logan (2017) reference, suggesting issues with the data presented in that reference. The above data is supported by the in vitro transwell data that suggested that 40:1 ratio is required at minimum.

However, a ratio of R8 to Lucentis of 200/1 enhanced delivery to the retina after 2 h (FIG. 14), see retinal Lucentis level in bottom graph, in comparison with equivalent graph in FIG. 12. FIG. 15 summaries the results of the in vivo assessment of Lucentis concentrations in the retina after topical application of R/L formulations at 2 h.

### Example 4—Hydrogel Formulation R8:Lucentis

To increase corneal contact time, R8:Lucentis formulations were made with hydrogels to enhance formulation viscosity and their properties were investigated. FIG. 16 shows that by increasing the concentration of a hydrogel in the formulation, a thermosensitive gel was formed with the desired properties. In the experiment shown in FIG. 16, Lutrol F127 was used as the hydrogel. Solutions were prepared by mixing the desired quantities of Lutrol F127 with 20 mM HEPES buffer, pH 7.4. Between 20% and 25% Lutrol was the optimal concentration range tested. The R8: Lucentis formulation was stable at 4° C. for seven days in the presence of increasing concentrations of Trehalose (FIG. 17A). In addition, reducing the Lucentis concentration whilst keeping the ratio of R8:Lucentis the same, aided stability at higher ratios. Furthermore the 20% Lutrol hydrogel improved the stability of Lucentis formulations. The presence of Lutrol helped prevent aggregation of R8:Lucentis formulations even at 200:1 ratio. FIG. 17B shows that a formulation of R8:Lucentis (50:1) and hydrogel (20% Lutrol) was stable for 12 weeks at 4° C. FIG. 17C shows that a formulation of R8:Lucentis (50:1) and hydrogel (20% Lutrol) remained stable for 12 hours at 4° C. and 25° C. FIG. 17D shows that a formulation of R8:Lucentis (50:1), hydrogel and the mucoadhesive polymer 5K chitosan (01% w/v) showed good stability for 4 weeks as determined by EC₅₀ ratio (U8:Lucentis/Standard) of less than 2.

FIG. 18A shows that a formulation of 20% Lutrol gel, 10 mm HisCl, 10% trehalose, 0.01% polysorbate 20, pH 5.5 took approximately 1 hr to release Lucentis in a release assay. FIG. 18B summaries the stability results for the R8/Lucentis liquid and hydrogel formulations. R8/Lucentis ratios of up to/including 100:1 in liquid formulation were stable in a concentration of 100 or 600 mg/ml Trehalose. In a 20% Lutrol hydrogel formulation R8/Lucentis ratios of up to/including 200:1 were stable in concentrations of 100-600 mg/ml Trehalose.

Incorporation of Lucentis/R8 into a hydrogel dramatically increased the concentration of anti-VEGF delivered to posterior ocular tissues of the rabbit, due to enhanced corneal contact time (FIG. 19). FIG. 19 shows that R8/Lucentis 200:1 ratio gel delivered significant concentrations of Lucentis to the retina at 2 h (A) and 12 h (B) post hydrogel administration. At both timepoints the Lucentis concentration exceeded the target concentration in the retina of at least 200 ng/g retinal tissue. The data is summarised in FIG. 19C. FIG. 20 shows that R8/Lucentis 50:1 ratio gel delivered significant concentrations of Lucentis to the retina at 2 h (A), 12 h (B) and 24 h (C) post hydrogel administration. At 2 h and 12 h timepoints the Lucentis concentration exceeded the target concentration in the retina of at least 200 ng/g retinal tissue. At 24 h post hydrogel administration the Lucentis concentration had dropped below the retinal target level of 200 ng/g retinal tissue. The data is summarised in FIG. 20D.

FIG. 21A summarises the in vivo assessment of Lucentis concentrations in the retina after topical application of R/L formulations at 2 h, 12 h, 24 h and 72 h, with the addition of a hydrogel and chitosan as indicated. A R8/Lucentis 80:1 ratio hydrogel formulation with 0.1% w/v chitosan seemed to give the best retinal Lucentis profile.

FIG. 21B shows that the preferred U8:Lucentis formulation (80:1 R8 to Lucentis ratio, hydrogel, trehalose and chitosan) is not associated with redness or corneal damage in vivo in rabbit eyes, as seen by fluorescein staining.

FIG. 22 summaries the experiments conducted, and ranges of R8/Lucentis ratios tested, to come to the conclusion that a ratio range of 50-80:1 R8 to Lucentis is the optimal ratio range.

### Example 5—Investigation into the Mechanism of R8/Lucentis Interaction

The interaction between R8 and Lucentis was investigated further. One hypothesis considered was that as R8 is highly charged, it may electrostatically associate with the protein surface giving the particle a net positive charge, so enhancing drug delivery across ocular membranes. Another hypothesis considered was the bystander effect. R8 could destabilize cell membranes through the interaction with lipid phosphate groups. R8 mediated disruption of the ocular tear film and corneal epithelial plasma membrane could thus enhance the passive diffusion of drugs into ocular tissues.

The first experiment conducted was Size Exclusion Chromatography (SEC), FIG. 23. There was no co-elution of of Lucentis and FITC-R8, so no binding. The second experiment conducted was Quartz Crystal Microbalance (QCM), FIG. 24. This method measures a mass variation per unit area by measuring the change in frequency of a quartz crystal resonator. The resonance is disturbed by the addition or removal of a small mass due to oxide growth/decay or film deposition at the surface of the acoustic resonator.

Δf=−C_{f}·Δm Sauerbrey Equation

Where Δf—the observed frequency change in Hz.

Δm—the change in mass per unit area, in g/cm², and

Cf—the sensitivity factor for the crystal used (i.e. 56.6 Hz μg⁻¹ cm² for a 5 MHz AT-cut quartz crystal at room temperature)

Attana QCM system; approx . . . 4.4 Hz/(ng*cm²) which further reduces to 0.7 Hz/ng (1.5 mm²)

75*0.7=52.5 ng (11.3 pmol of Lucentis bound)

Hypothetical calculations: 1 mm depth=1.5 mm³ (1.5 μl)

20 μM R8 in 1.5 mm³=30 pmol R8 (R8:Lucentis=2.6)

200 μM R8 in 1.5 mm³=300 pmol R8 (R8:Lucentis=26)

1 μL/s flow rate, 20 μM (0.26-2.6):1 ratio, 200 μM (2.6-26) ratio R8: Lucentis, control: 4Hx difference=5.7 ng VEGF 165 (6.25 μg added).

Lucentis was immobilised on a chip surface and tested, no binding to R8 was detected.

The third experiment conducted was Surface Plasmon Resonance (SPR), FIG. 25. Lucentis was immobilised on a CMS chip surface RU=3700. Increasing concentrations of R8 we tested either in buffer of 10 mM HisCl pH 5.5, 10% Trehalose or 10 mM HEPES, pH 7.4, 150 mM NaCl, 3 mM EDTA and 0.05% v/v surfactant P20. Some binding of R8 to Lucentis was detected.

The fourth experiment conducted was Isothermal titration calorimetry (ITC) (FIG. 26). This is a technique used to determine the thermodynamic parameters of interactions in solution. It is most often used to study the binding of small molecules (such as medicinal compounds) to larger macromolecules (proteins, DNA etc.) ITC is a quantitative technique that can determine the biding affinity, enthalpy changes and binding stoichiometry of the interaction between two or more molecules on solution. From these initial measurements, Gibbs energy changes and entropy changes can be determined using the relationship; dG=−RTInKa=dH−TdS. No binding between R8 and Lucentis was detected at ratios up to 180 R8: Lucentis.

The fifth experiment conducted was whether R8 affected the transport of dextrans across a membrane, FIG. 27. Dextrans are complex branched polysaccharides made of many glucose molecules. They can be composed of chains of varying lengths. In a dextran transcytosis assay, used to check if R8 carried dextrans of various sizes across the membrane, R8 made no difference to the transport of the different sizes of dextran.

The results of the experiments are summarised in FIG. 28. Due to the lack of evidence for binding to Lucentis, it seems that R8 may be working via the bystander effect. If so, then the action of R8 would aid in the transport of a variety of macromolecules and drugs across membranes.

### Example 6—Exemplified Product Specification and Method of Manufacture

### HICF Product Preparation

### Day 1

1. Buffer A preparation: 20% Lutrol F127, 0.1% w/v 5k chitosan, 20 mg/mL R8 peptide:

a. To 40 mL of pre-chilled (4° C.) distilled water add the following with mixing (4° C. roller for 1-3 h);

| Constituent | Molecular weight (Da) | Amount required | |
|---|---|---|---|
| Lutrol F127 | 15000 | 10 | g |
| 5k Chitosan | 5000 | 50 | mg |
| R8 peptide | 1267.51 | 500 | mg |

b. Make up to 50 mL with additional chilled distilled water

c. Mix 50 mL of Buffer A with 50 mL of Lucentis (as provided) 4° C. roller for 1 h

d. 0.2 μm filter the resulting solution and store at 4° C. until required (ideally use immediately)

e. Prepare 2 mL aliquots of this material into 6 mL glass vials²

2. Freeze-drying (conditions will require optimisation—seeks to achieve a ×2 fold concentration of product)

a. Freezing: −60° C., 2 h

b. Primary Drying: −34° C., 100 mTorr, 18 h

c. Secondary Drying: 20° C., 100 mTorr, 2 h

d. Store cakes at 4° C. until required, protecting from light

### Day 2

3. Rehydration (performed in 4° C. cold room)

a. To a freeze-dried cake (prepared in 1-2 above) add 1 mL (half-pre-freeze drying volume to give a two-fold concentrating effect) of pre-chilled sterile distilled water. Rehydrate with rotation (tube roller 2 h at 4° C. and 30 rpm or until cake is fully dissolved).

b. 0.2 μm filter the resulting solution and aliquot into vials.

c. Approximate Final composition (may be additional dilution effects due to high solute concentration)
10 mg/mL Lucentis
20 mg/mL R8
20% Lutrol F127
10 mM histidine HCl
10% w/v α,α-trehalose dihydrate
0.01% w/v polysorbate 20
0.1% w/v 5k Chitosan
pH 5.5
NOTES

1. Suggested filter type: Millipore® Stericup™ filter units PVDF membrane (Durapore), very low protein binding, pore size 0.22 μm, funnel capacity 150 mL CAT NO: #SCGVU01RE, available from http://www.merckmillipore.com/GB/en/product/Stericup-GV-Sterile-Vacuum-Filtration-System,MM_NF-SCGVU01RE
2. Alternative is to freeze-dry in bulk and rehydrate in bulk before sterile filtering and aliquotting the resulting solution

### Example 7—Appendix SOP for Lucentis Activity ELISA

### Day 1

### Plate Coating

1. Defrost one aliquot of hVEGF₁₆₅ per plate and add 5.40 mL of coating buffer to the Eppendorf tube (0.25 μg/mL)

2. Coat each well of a 96 well plate with 50 μL/well of the hVEGF₁₆₅ solution

3. Tap the plate to ensure good coverage before covering and incubating at 4° C. overnight

### Day 2

### Blocking (2 h, Prepare Lucentis Dilutions in the Gap)

1. Wash plate three times with 300 μL/well wash buffer. Each wash should be left on the plate for 5 minutes

2. Block non-specific binding sites with 300 μL/well of blocking buffer. Incubate the sealed plate for 1.5 h with shaking.

### Lucentis Dilutions (2 h, Based on ScFv Dilution Protocol)

| | |
|---|---|
| Dilution | To 50 mL of PBS add 0.5 g BSA and 100 μL of Tween 20. |
| buffer: | Mix at room temperature to dissolve and filter (0.2 μm) |
| | before use. |

1. Wash plate three times with 300 82 L/well wash buffer. Each wash should be left on the plate for 5 minutes

2. For the standard curve, prepare the following dilutions in dilution buffer or matrix;
a. Dilute 0.05 mL Lucentis stock solution (10 mg/mL) into 4.95 mL of dilution buffer [Solution A, 100 μg/mL]
b. Dilute 0.05 mL of [Solution A] into 4.95 mL of dilution buffer [Solution B, 1000 ng/mL]
c. Dilute 0.05 mL of [Solution A] into 4.95 mL of dilution buffer with 1 μg/mL of R8 (5 μL of 1 mg/mL stock solution) [Solution B-R, 1000 ng/mL]
d. Dilute 0.15 mL of [Solution B] into 1.35 mL of Dilution buffer [Solution 1, 100 ng/mL](make ×4)
e. Dilute 0.15 mL of [Solution B-R] into 1.35 mL of Dilution buffer [Solution 1-R, 100 ng/mL](make ×4)
f. From Solutions 1 and 1-R prepare the following dilution range (×4 each);

| | Lucentis | Volume of stock | | Volume of dilutant |
|---|---|---|---|---|
| Solution | (ng/mL) | (μL) | Stock | (μL) |
| 1 | 100 | 750 | 1 | 0 |
| 2 | 60 | 420 | 1 | 280 |
| 3 | 40 | 400 | 1 | 200 |
| 4 | 20 | 250 | 3 | 250 |
| 5 | 10 | 250 | 4 | 250 |
| 6 | 5 | 250 | 5 | 250 |
| 7 | 1 | 100 | 6 | 400 |
| 8 | 0.2 | 100 | 7 | 400 |

g. Add 50 μL of each to the plate and incubate for 2 h with gentle shaking.

| | 1 2 3 | | 4 5 6 | | 7 8 9 | | 10 11 12 | |
|---|---|---|---|---|---|---|---|---|
| A | 100 | ng/mL | 100 | ng/mL | 100 | ng/mL | 100 | ng/mL |
| B | 60 | ng/mL | 60 | ng/mL | 60 | ng/mL | 60 | ng/mL |
| C | 40 | ng/mL | 40 | ng/mL | 40 | ng/mL | 40 | ng/mL |
| D | 20 | ng/mL | 20 | ng/mL | 20 | ng/mL | 20 | ng/mL |
| E | 10 | ng/mL | 10 | ng/mL | 10 | ng/mL | 10 | ng/mL |
| F | 5 | ng/mL | 5 | ng/mL | 5 | ng/mL | 5 | ng/mL |
| G | 1 | ng/mL | 1 | ng/mL | 1 | ng/mL | 1 | ng/mL |
| H | 0.2 | ng/mL | 0.2 | ng/mL | 0.2 | ng/mL | 0.2 | ng/mL |

### Antibody Coating (1.5 h)

3. Wash plate three times with 300μL/well wash buffer. Each wash should be left on the plate for 5 minutes

| | |
|---|---|
| Anti-human | From lyophilised stock |
| HRP IgG: | Rehydrate peroxidase-labelled anti-human IgG antibody |
| | with 1 mL of 50% glycerol solution to yield a |
| | concentration of 0.1 mg/mL. Solution is stable a 4° C. |
| | for 1 year. |
| | Working solution (From avastin assay) |
| | Add 3 μL of antibody stock solution to 10 mL of |
| | dilution buffer, mix well before use. |

4. Add 100 μL of anti-human HRP-IgG to each well, react for 1 h at room temperature

### Plate Development (1 h)

5. Wash plate three times with 300 μL/well wash buffer. Each wash should be left on the plate for 5 minutes

6. Turn off the lights

7. Add 50 μL/well POD substrate

8. Incubate for 15 minutes

9. Stop the reaction by adding 50 μL/well of 1M HCl

10. Measure absorbance at 450 nm against a reference wavelength of 690 nm

Lucentis activity note: http://www.ema.europa.eu/docs/en_GB/document_library/EPAR_-Scientific_Discussion/human/000715/WC500043550.pdf

“Ranibizumab dose-dependently inhibited VEGF-induced proliferation in human umbilical vein endothelial cell (HUVEC), which are cells that express the VEGF-receptors, with IC50 values below 1 nM. This is 10-20-fold over clinical Cmax. Roughly approximated IC10 levels were somewhat higher than the clinical Cmax values (1.7 ng/mL predicted at steady state, maximal individual level 2.4 ng/mL) and consequently, no significant systemic VEGF-inhibiting activity is expected in humans. A total inhibition of proliferation was observed at ranibizumab concentrations≥1.3 nM. Ranibizumab inhibited rhVEGF165-induced tissue factor up-regulation in a dose-dependent manner, with an IC50 of 0.31 nM.”

### REFERENCES

F. de Cogan, L. J. Hill, A. Lynch, P. J. Morgan-Warren, J. Lechner, M. R. Berwick, A. F. A. Peacock, M. Chen, R. A. H. Scott, H. Xu, A. Logan, Investig. Opthalmology Vis. Sci. 2017, 58, 2578.

B. Davis, E. Normando, L. Guo, P. O'Shea, S. Moss, S. Somavarapu, M. Cordeiro, Small 2014, 10, 1575.

F. Oling, W. Bergsma-Schutter, A. Brisson, J. Struct. Biol. 2001, 133, 55.

B. Campos, S. Wang, G. S. Retzinger, M. A. Kaetzel, B. A. Seaton, N. J. Karin, J. D. Johnson, J. R. Dedman, Arch. Med. Res. n.d., 30, 360.

Y. Mo, B. Campos, T. R. Mealy, L. Commodore, J. F. Head, J. R. Dedman, B. A. Seaton, J. Biol. Chem. 2003, 278, 2437.

V. Kheifets, R. Bright, K. Inagaki, D. Schechtman, D. Mochly-Rosen, J. Biol. Chem. 2006, 281, 23218.

M. Jin, C. Smith, H.-Y. Hsieh, D. F. Gibson, J. F. Tait, 2004, DOI 10.1074/jbc.M405846200.

M. Vazdar, E. Wernersson, M. Khabiri, L. Cwiklik, P. Jurkiewicz, M. Hof, E. Mann, S. Kolusheva, R. Jelinek, P. Jungwirth, J. Phys. Chem. B 2013, 117, 11530.

K. Melikov, A. Hara, K. Yamoah, E. Zaitseva, E. Zaitsev, L. V Chernomordik, Biochem. J 2015, 471, 221.

Z. Wu, Q. Cui, A. Yethiraj, J. Phys. Chem. B 2013, 117, 12145.

H. Kenis, H. van Genderen, N. M. Deckers, P. a G. Lux, L. Hofstra, J. Narula, C. P. M. Reutelingsperger, Exp. Cell Res. 2006, 312, 719.

H. Kenis, H. van Genderen, A. Bennaghmouch, H. a Rinia, P. Frederik, J. Narula, L. Hofstra, C. P. M. Reutelingsperger, J. Biol. Chem. 2004, 279, 52623.

J. H. Trueblood, R. M. Rossomondo, L. A. Wilson, W. H. Carlton, Arch. Ophthalmol. (Chicago, Ill. 1960) 1975, 93, 127.

Y. Saishin, Y. Saishin, K. Takahashi, R. L. e Silva, D. Hylton, J. S. Rudge, S. J. Wiegand, P. A. Campochiaro, J. Cell. Physiol. 2003, 195, 241.

L. Yu, X. Wu, Z. Cheng, C. V. Lee, J. LeCouter, C. Campa, G. Fuh, H. Lowman, N. Ferrara, IOVS 2008, 49, 522.

H. Gerber, X. Wu, L. Yu, C. Wiesmann, X. Liang, C. Lee, G. Fuh, C. Olsson, L. Damico, D. Xie, Y. Meng, J. Gutierrez, R. Corpuz, B. Li, L. Hall, L. Rangell, R. Ferrando, H. Lowman, F. Peale, N. Ferrara, PNAS 2007, 104, 3478.

W.-C. Liang, X. Wu, F. V. Peale, C. V. Lee, Y. G. Meng, J. Gutierrez, L. Fu, A. K. Malik, H.-P. Gerber, N. Ferrara, G. Fuh, J. Biol. Chem. 2006, 281, 951.

C. Fischak, et al. J. Ophthalmology 2017, Article ID 5192924, 6 pages

D. Schmidl, et al., J. Ocular Pharmacology and Therapeutics, 2017, 33, 375-382.

1. A composition comprising a peptide which is RRRRRRRR (SEQ ID NO: 1) and Ranibizumab, wherein the molar ratio of the peptide to Ranibizumab is 40:1 to 200:1.

2-7. (canceled)

8. The composition of claim 1, wherein the molar ratio of the peptide to Ranibizumab is 50:1 to 200:1.

9. The composition of claim 8, wherein the molar ratio of the peptide to Ranibizumab is 50:1 to 80:1.

10. The composition of claim 1, wherein the composition also comprises Trehalose.

11. The composition of claim 10, wherein Trehalose is present at a concentration of 100 mg/ml to 600 mg/ml.

12. (canceled)

13. The composition of claim 1, wherein the composition also comprises a hydrogel.

14. The composition of claim 13, wherein the hydrogel concentration is between 15% and 25% w/v, optionally between 20% to 25% w/v.

15. (canceled)

16. The composition of claim 1, wherein the composition also comprises chitosan.

17. The composition of claim 16, wherein the chitosan concentration is between 0.05% and 0.25% w/v.

18. The composition of claim 17, wherein the chitosan concentration is 0.1% w/v.

19. A method of treatment of a condition characterised by VEGF dysfunction, said method comprising administering a prophylactically or therapeutically effective amount of the composition as defined in claim 1 to a patient in need thereof.

20. The method of claim 19, wherein the condition characterised by VEGF dysfunction is cancer, oedema or leaky vessels.

21. The method of claim 19, wherein the condition characterised by VEGF dysfunction is a vasculoproliferative condition.

22. The method of claim 21, wherein the vasculoproliferative condition comprises neovascularisation, vascular endothelial cell proliferation, angiogenesis, telangiectasia or microaneurysms.

23. A method of treatment of a vasculoproliferative condition of the eye, said method comprising administering a prophylactically or therapeutically effective amount of the composition as defined in claim 1 to a patient in need thereof.

24. The method of claim 23, wherein the vasculoproliferative condition of the eye is selected from age-related macular degeneration (AMD), diabetic retinopathy, retinal vein occlusion, retinopathy of prematurity, macular telangiectasia, or choroidal neovascularisation, diabetic vasculopathy, diseases characterised by retinal angiogenesis and sickle cell disease.

25. The method of claim 19, wherein the composition is formulated for topical administration.

26. The method of claim 25, wherein the composition is formulated for topical administration to the eye.

27. The method of claim 26, wherein the composition is administered to the eye by eye drop.

28-30. (canceled)

31. A kit comprising the composition as defined in claim 1.

32. The method of claim 23, wherein the composition is formulated for topical administration.


SEQUENCE LISTING (1 sequence)

SEQ ID NO: 1 - protein, 8 aa, Artificial Sequence
  R8 peptide
RRRRRRRR